(19) United States
(12) Patent Application Publication
(10) Pub. No.: US 2003/0236288 A1
(43) Pub. Date: Dec. 25, 2003

(54) Use of substituted 3-phenyl-5-alkoxy-3H-(1,3,4)-oxadizol-2-ones for inhibiting pancreatic lipase

(76) Inventors: Karl Schoenafinger, Alzenau (DE); Stefan Petry, Frankfurt (DE); Guenter Mueller, Sulzbach (DE); Armin Bauer, Sulzbach (DE); Hubert Otto Heuer, Schwabenheim (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/376,579

(22) Filed: Feb. 28, 2003

Related U.S. Application Data
(60) Provisional application No. 60/365,704, filed on Mar. 19, 2002.

(30) Foreign Application Priority Data
Feb. 28, 2002 (DE) 10208986.8

(51) Int. Cl. A61K 31/4245

(52) U.S. Cl. 514/364

(57) ABSTRACT

The invention relates to a method for inhibiting pancreatic lipase, or the prophylaxis or treatment of obesity or diabetes mellitus of type 1 and 2, in a patient in need thereof, comprising administering to the patient a pharmaceutically effective amount of substituted 3-phenyl-5-alkoxy-3H-(1,3,4)-oxadiazol-2-ones of formula 1:

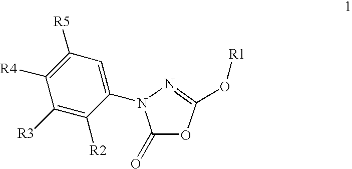

wherein R₁, R₂, R₃, R₄ and R₅ are as defined herein, or a prodrug, solvate, pharmacologically acceptable salt or acid addition salt thereof.

### FIELD OF THE INVENTION

The invention relates to a method for inhibiting pancreatic lipase, in a patient in need thereof, comprising administering to the patient a pharmaceutically effective amount of a substituted 3-phenyl-5-alkoxy-3H-(1,3,4)-oxadiazol-2-one. The invention also relates to a method for the prophylaxis or treatment of obesity or diabetes mellitus of type 1 and 2, in a patient in need thereof, comprising administering to the patient a pharmaceutically effective amount of a substituted 3-phenyl-5-alkoxy-3H-(1,3,4)-oxadiazol-2-one.

### BACKGROUND OF THE INVENTION

Substituted 3-phenyl-5-alkoxy-3H-(1,3,4)-oxadiazol-2-ones with an inhibitory effect on hormone-sensitive lipase are disclosed in WO 01/17981 and WO 01/66531. The use of substituted 3-phenyl-5-alkoxy-3H-(1,3,4)-oxadiazol-2-ones as inhibitors on pancreatic lipase, PL, is not disclosed.

### SUMMARY OF THE INVENTION

The invention therefore relates to a method for inhibiting pancreatic lipase, in a patient in need thereof, comprising administering to the patient a pharmaceutically effective amount of a substituted 3-phenyl-5-alkoxy-3H-(1,3,4)-oxadiazol-2-one of formula 1:

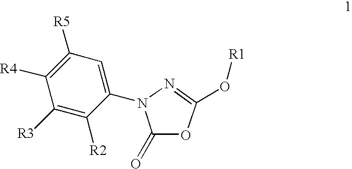

wherein:

R¹ is C₁-C₆-alkyl, or C₃-C₉-cycloalkyl, wherein the alkyl is optionally substituted one or more times by:

hydroxy;

fluorine;

phenyl, optionally substituted one or more times by halogen,

C₁-C₉-alkyl, C₁-C₈-alkyloxy, nitro, or CF₃;

C₁-C₄-alkyloxy;

C₁-C₄-alkyl-S—; or

(C₁-C₄-alkyl)₂N—; and

the cycloalkyl is optionally substituted one or more times by:

C₆-C₁₀ aryl, optionally substituted one or more times by halogen,

C₁-C₉-alkyl, C₁-C₈-alkyloxy, nitro, or CF₃;

C₁-C₄-alkyl;

C₁-C₄-alkyloxy;

C₁-C₄-alkyl-S—; or (C₁-C₄-alkyl)₂N—;

R², R³, R⁴ and R⁵ are each, independently,

hydrogen;

halogen;

NO₂;

C₁-C₄-alkyl;

C₁-C₉-alkyloxy, substituted one or more times by fluorine, hydroxy, C₆-C₁₀-aryl, amino, C₁-C₄-alkyl-NH— or (C₁-C₆-alkyl)₂N—;

C₆-C₁₀-aryl-C₁-C₄-alkyloxy, C₆-C₁₀-aryloxy, C₆-C₁₀-aryl, C₆-C₁₀-aryloxy-C₃-C₄-alkyl, C₃-C₈-cycloalkyl or C₃-C₈-cycloalkyloxy, wherein the alkyl is optionally substituted one or more times by halogen, hydroxy, CF₃, (C₁-C₆-alkyl)₂N—, C₁-C₄-alkyloxy or C₁-C₄-alkyl, the aryl is optionally substituted one or more times by halogen, CF₃, C₁-C₈-alkyloxy or C₁-C₉-alkyl, and the cycloalkyl is optionally substituted one or more times by halogen, CF₃, C₁-C₄-alkyloxy, C₆-C₁₀-aryl or C₁-C₄-alkyl;

C₁-C₆-alkyl-NH—SO₂—, wherein the alkyl is optionally substituted by hydroxy, fluorine or (C₁-C₆-alkyl)₂N—;

(2,2,6,6-tetramethylpiperidin-4-yl)-NH—SO₂—;

C₃-C₈-cycloalkyl-NH—SO₂—, wherein the cycloalkyl is optionally substituted one or more times by C₁-C₄-alkyl or C₆-C₁₀-aryl;

(C₁-C₆-alkyl)₂—N—SO₂—;

XCO—;

YSO₂—;

2-oxo-pyrrolidin-1-yl;

2,5-dimethylpyrrol-1-yl; or

R⁷-A-NR⁶—,

provided that R², R³, R⁴ and R⁵ are not simultaneously hydrogen;

X is C₁-C₆-alkyloxy;

C₁-C₆-alkyl-NH—;

C₃-C₈-cycloalkyl-NH—;

(C₁-C₆-alkyl)₂N—; or

1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;

Y is 1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;

R⁶ is hydrogen, C₁-C₄-alkyl or C₆-C₁₀-aryl-C₁-C₄-alkyl, wherein the aryl is optionally substituted by halogen, CF₃, C₁-C₈-alkyloxy or C₁-C₉-alkyl;

A is a single bond, —CO—, —O—C(O)—, —SOₙ— or —NR⁸C(O)—;

n is 1 or 2;

R⁷ is hydrogen;

C₁-C₁₈-alkyl or C₂-C₁₈-alkenyl, wherein the alkyl and alkenyl are optionally substituted once to three times by:

C₁-C₄-alkyl;

halogen;

hydroxy;

CF₃;

C₁-C₄-alkyloxy;

(C₁-C₄-alkyl)₂N—;

—COOH;

C₁-C₄-alkyloxycarbonyl;

oxo; or

C₆-C₁₂-aryl, C₆-C₁₂-aryloxy, C₆-C₁₂-arylcarbonyl or C₆-C₁₀-aryl-C₁-C₄-alkyloxy, wherein the aryl is optionally substituted by halogen, C₁-C₉-alkyl, C₁-C₈-alkyloxy, CF₃, aminosulfonyl or methylmercapto;

C₆-C₁₀-aryl-C₁-C₄-alkyl, C₅-C₈-cycloalkyl-C₁-C₄-alkyl, C₅-C₈-cycloalkyl, C₆-C₁₀-aryl-C₂-C₆-alkenyl, C₆-C₁₀-aryl, biphenylyl, biphenylyl-C₁-C₄-alkyl or indanyl, wherein the alkyl, aryl, cycloalkyl, alkenyl, biphenyl and indanyl are each independently optionally substituted one or more times by:

C₁-C₁₈-alkyl, C₁-C₁₈-alkyloxy, C₃-C₈-cycloalkyl, C₁-C₄-alkylcarbonyl, C₆-C₁₀-aryl-C₁-C₄-alkyl, C₆-C₁₀-aryl-C₁-C₄-alkyloxy or C₁-C₆-alkyloxycarbonyl, wherein the alkyl is optionally substituted by fluorine, hydroxy, (C₁-C₄-alkyl)₂N—, C₁-C₄-alkyloxycarbonyl, CF₃ or carboxyl, and the aryl is optionally substituted by halogen, CF₃, C₁-C₉-alkyl or C₁-C₈-alkyloxy;

COOH;

hydroxy;

(C₁-C₄-alkyl)₂N—;

C₆-C₁₀-aryloxy, optionally substituted by C₁-C₉-alkyl, C₁-C₈-alkyloxy, halogen or CF₃;

NO₂;

NC—;

C₆-C₁₀-aryl, optionally substituted by C₁-C₉-alkyl, C₁-C₈-alkyloxy, halogen or CF₃;

fluorosulfonyl;

H₂NSO₂—;

C₁-C₄-alkylcarbonyloxy;

C₆-C₁₀-arylsulfonyloxy;

pyridyl;

C₆-C₁₀-aryl-SO₂NH—;

halogen;

CF₃; or

OCF₃; or

Het-(CH₂)ᵣ—, wherein r is 0, 1, 2 or 3 and Het is saturated or unsaturated 5 to 7-membered heterocycle that is optionally benzo-fused, wherein the heterocycle portion is optionally substituted by:

C₁-C₄-alkyl;

C₆-C₁₀-aryl, optionally substituted by C₁-C₉-alkyl, C₁-C₈-alkyloxy, halogen or CF₃;

halogen;

NO₂;

C₁-C₄-alkyloxy;

C₁-C₄-alkyloxycarbonyl; or

C₆-C₁₀-aryl-C₁-C₄-alkyl or C₆-C₁₀-aryl-C₁-C₄-alkylmercapto, wherein the alkyl is optionally substituted by hydroxy, (C₁-C₄-alkyl)₂N—, fluorine, methoxy or CF₃, and the aryl is optionally substituted by C₁-C₉-alkyl, C₁-C₈-alkyloxy, halogen or CF₃;

and wherein the benzo portion is optionally substituted by halogen, C₁-C₄-alkyloxy or CF₃; and

R⁸ is hydrogen or C₁-C₄-alkyl; or a prodrug, solvate, pharmacologically acceptable salt, or acid addition salt thereof.

### DETAILED DESCRIPTION OF THE INVENTION

Definition of Terms

Halogen is fluorine, chlorine or bromine, preferably fluorine or chlorine.

Alkyl, alkenyl and alkyloxy as used herein may be branched or unbranched.

5 to 7-membered heterocycle as used herein is, for example, furan, thiophene, isoxazole, pyridine, piperidine, piperizine and pyrrolidine.

Patient includes both human and other mammals.

Pharmaceutically effective amount means an amount of the compound according to the invention effective in producing the desired therapeutic effect.

Particular or Preferred Embodiment

A particular method for inhibiting pancreatic lipase is administering a compound of formula 1 wherein:

wherein:

R¹ is C₁-C₆-alkyl, or C₃-C₉-cycloalkyl, wherein the alkyl and cycloalkyl are each independently optionally substituted one or more times by:

phenyl, optionally substituted one or more times by halogen, C₁-C₄-alkyl, C₁-C₄-alkyloxy, nitro, or CF₃;

C₁-C₄-alkyloxy;

C₁-C₄-alkyl-S—; or

(C₁-C₄-alkyl)₂N—.

A particular method for inhibiting pancreatic lipase is administering a compound of formula 1 wherein:

R², R³, R⁴ and R⁵ are each, independently, hydrogen;

halogen;

NO₂;

C₁-C₄-alkyl;

C₁-C₉-alkyloxy, substituted one, two or three times by fluorine, C₆-C₁₀-aryl, amino, C₁-C₄-alkyl-NH— or (C₁-C₆-alkyl)₂N—;

C₆-C₁₀-aryl-C₁-C₄-alkyloxy, C₆-C₁₀-aryloxy, C₆-C₁₀-aryl, C₆-C₁₀-aryloxy-C₃-C₄-alkyl, C₃-C₈-cycloalkyl or C₃-C₈-cycloalkyloxy, wherein the aryl, alkyl and cycloalkyl are each independently optionally substituted one, two or three times by halogen, CF₃, C₁-C₄-alkyloxy or C₁-C₄-alkyl;

C₁-C₆-alkyl-NH—SO₂—, wherein the alkyl is optionally substituted by (C₁-C₆-alkyl)₂N—;

(2,2,6,6-tetramethylpiperidin-4-yl)-NH—SO₂—;

C₃-C₈-cycloalkyl-NH—SO₂—, wherein the cycloalkyl is optionally substituted one or more times by C₁-C₄-alkyl;

(C₁-C₆-alkyl)₂—N—SO₂—;

XCO—; YSO₂—;

2-oxo-pyrrolidin-1-yl;

2,5-dimethylpyrrol-1-yl; or

R⁷-A-NR⁶

provided that R², R³, R⁴ and R⁵ are not simultaneously hydrogen.

A particular method for inhibiting pancreatic lipase is administering a compound of formula 1 wherein: A is a single bond, —CO—, —O—C(O)—, —SOₙ— or —NHC(O)—, wherein n is 1 or 2.

A particular method for inhibiting pancreatic lipase is administering a compound of formula 1 wherein:

R⁷ is hydrogen;

C₁-C₁₈-alkyl or C₂-C₁₈-alkenyl, wherein the alkyl and alkenyl are each independently optionally substituted once to three times by:

C₁-C₄-alkyl;

halogen;

CF₃;

C₁-C₄-alkyloxy;

(C₁-C₄-alkyl)₂N—;

—COOH;

C₁-C₄-alkyloxycarbonyl;

oxo; or

C₆-C₁₂-aryl, C₆-C₁₂-aryloxy, C₆-C₁₂-arylcarbonyl or C₆-C₁₀-aryl-C₁-C₄-alkyloxy, wherein the aryl is optionally substituted by halogen, C₁-C₄-alkyl, C₁-C₄-alkyloxy, aminosulfonyl or methylmercapto;

C₆-C₁₀-aryl-C₁-C₄-alkyl, C₅-C₈-cycloalkyl-C₁-C₄-alkyl, C₅-C₈-cycloalkyl, C₆-C₁₀-aryl-C₂-C₆-alkenyl, C₆-C₁₀-aryl, biphenylyl, biphenylyl-C₁-C₄-alkyl or indanyl, wherein the alkyl, aryl, cycloalkyl, alkenyl, biphenyl and indanyl are each independently optionally substituted once or twice by:

C₁-C₁₈-alkyl, C₁-C₁₈-alkyloxy, C₃-C₈-cycloalkyl, C₁-C₄-alkylcarbonyl, C₆-C₁₀-aryl-C₁-C₄-alkyl, C₆-C₁₀-aryl-C₁-C₄-alkyloxy or C₁-C₆-alkyloxycarbonyl, wherein the alkyl is optionally substituted by C₁-C₄-alkyloxycarbonyl, CF₃ or carboxyl, and the aryl is optionally substituted by halogen, CF₃, or C₁-C₄-alkyloxy;

COOH;

hydroxy;

C₆-C₁₀-aryloxy;

NO₂;

NC—;

C₆-C₁₀-aryl;

fluorosulfonyl;

C₆-C₁₀-arylsulfonyloxy;

pyridyl;

C₆-C₁₀-aryl-SO₂NH—;

halogen;

CF₃; or

OCF₃; or

Het-(CH₂)ᵣ—, wherein r is 0, 1, 2 or 3 and Het is saturated or unsaturated 5 to 7-membered heterocycle that is optionally benzo-fused, wherein the heterocycle portion is optionally substituted by:

C₁-C₄-alkyl;

C₆-C₁₀-aryl;

halogen;

NO₂;

C₁-C₄-alkyloxy;

C₁-C₄-alkyloxycarbonyl; or

C₆-C₁₀-aryl-C₁-C₄-alkyl or C₆-C₁₀-aryl-C₁-C₄-alkylmercapto, wherein the

alkyl is optionally substituted by methoxy or CF₃;

and wherein the benzo portion is optionally substituted by halogen, C₁-C₄-alkyloxy or CF₃.

A preferred method for inhibiting pancreatic lipase is administering a compound of formula 1 wherein: R¹ is C₁-C₆-alkyl, optionally substituted by phenyl.

A preferred method for inhibiting pancreatic lipase is administering a compound of formula 1 wherein: R⁵ is hydrogen.

A preferred method for inhibiting pancreatic lipase is administering a compound of formula 1 wherein: R² is hydrogen, halogen, C₁-C₄-alkyl, C₁-C₉-alkyloxy or amino.

A further preferred method for inhibiting pancreatic lipase is administering a compound of formula.1 wherein:

R³ is hydrogen;

C₁-C₄-alkyl;

C₆-C₁₀-aryl-C₁-C₄-alkyloxy, wherein the aryl is optionally substituted by halogen; or R⁷-A-NR⁶;

R⁶ is hydrogen or benzyl;

A is single bond; and

R⁷ is C₆-C₁₀-aryl-C₁-C₄-alkyl, wherein the aryl and alkyl are each independently optionally substituted by halogen, CF₃, cyano, phenyl-C₁-C₄-alkyloxy, CF₃-phenoxy, C₅-C₈-cycloalkyl or fluorosulfonyl;

C₁-C₁₂-alkyl, optionally substituted by C₁-C₄-alkyloxy, phenyl, CF₃ or phenyl-C₁-C₄-alkyloxy;

C₂-C₁₂-alkenyl; or Het-(CH₂)ᵣ—, wherein r is 0 or 1, and Het is saturated or unsaturated 5 to 7-membered heterocycle that is optionally benzo-fused and wherein the heterocyle portion is optionally substituted by C₁-C₄-alkyl or halogen.

A further preferred method for inhibiting pancreatic lipase is administering a compound of formula 1 wherein:

R² and R³ are each, independently,

hydrogen;

C₆-C₁₀-aryl;

C₃-C₈-cycloalkyl;

optionally C₁-C₄-alkyl-substituted C₆-C₁₀-aryloxymethyl;

optionally mono- or poly-C₁-C₄-alkyl- or halogen-substituted benzyloxy, C₆-C₁₀-aryloxy or C₃-C₈-cycloalkyloxy; mono- or poly-fluorine-, C₆-C₁₀-aryl- or amino-substituted C₁-C₆-alkyloxy,

wherein the amino is optionally substituted once or twice by C₁-C₄-alkyl;

C₁-C₆-alkyl-NH—SO₂—, wherein the alkyl is optionally substituted by (C₁-C₆-alkyl)₂N—;

(2,2,6,6-tetramethylpiperidin-4-yl)-NH—SO₂—; C₃-C₈-cycloalkyl-NH—SO₂—, wherein the cycloalkyl is optionally substituted by C₁-C₄-alkyl;

(C₁-C₆-alkyl)₂—N—SO₂—;

YSO₂—, wherein Y is 1-piperidinyl, 4-morpholinyl or 1-piperazinyl, wherein the piperidinyl, morpholinyl and piperazinyl are each independently optionally substituted by C₁-C₄-alkyl;

XCO—, wherein X is (C₁-C₆-alkyl)₂N—, 1-piperidinyl, 4-morpholinyl or 1-piperazinyl, wherein the piperidinyl, morpholinyl and piperazinyl are each independently optionally substituted by C₁-C₄-alkyl.

An additionally preferred method for inhibiting pancreatic lipase is administering a compound of formula 1 wherein:

R⁴ is hydrogen;

2-oxo-pyrrolidin-1-yl;

2,5-dimethylpyrrol-1-yl; or

C₆-C₁₀-aryl-C₁-C₄-alkyloxy, wherein the aryl and alkyl are each independently optionally substituted by halogen.

An additionally preferred method for inhibiting pancreatic lipase is administering a compound of formula 1 wherein:

R⁴ is R⁷-A-NR⁶;

R⁶ is hydrogen or methyl;

A is single bond; and

R⁷ is hydrogen;

C₁-C₁₂-alkyl, optionally substituted once or twice by halogen;

C₂-C₁₈-alkenyl, optionally substituted once or twice by C₁-C₄-alkyl or C₁-C₄-alkyloxycarbonyl;

C₆-C₁₀-aryl-C₁-C₄-alkyl, wherein the alkyl and aryl are each independently optionally substituted by:

halogen;

C₁-C₆-alkyloxy;

CF₃;

NC—;

C₅-C₆-cycloalkyl;

C₁-C₄-alkyloxycarbonyl;

C₆-C₁₀-aryl-C₁-C₄-alkyl or C₆-C₁₀-aryl-C₁-C₄-alkyloxy, wherein the aryl is optionally substituted by halogen or CF₃;

C₅-C₈-cycloalkyl-C₁-C₄-alkyl; or

Het-(CH₂)ᵣ—, wherein r is 1, 2 or 3 and Het is saturated or unsaturated 5 to 7-membered heterocycle, optionally substituted by halogen, C₁-C₄-alkyloxy or C₁-C₄-alkyloxycarbonyl,

An additionally preferred method for inhibiting pancreatic lipase is administering a compound of formula I wherein:

R⁴ is R⁷-A-NR⁶;

R⁶ is hydrogen;

A is —CO—; and

R⁷ is C₁-C₁₈-alkyl, optionally substituted by:

halogen;

phenyl;

phenoxy, optionally substituted by methyl, halogen or

methylmercapto;

phenylcarbonyl; or

C₁-C₄-alkyloxycarbonyl;

C₂-C₁₈-alkenyl, optionally substituted by C₆-C₁₀-aryl;

C₆-C₁₀-aryl, optionally substituted by:

halogen;

C₁-C₈-alkyl;

phenyl-C₁-C₄-alkyl;

CF₃;

OCF₃;

fluorosulfonyl;

C₁-C₄-alkyloxycarbonyl; or

phenoxy, optionally substituted by C₁-C₄-alkyloxy;

C₆-C₁₀-aryl-C₁-C₄-alkyl, wherein the alkyl is optionally substituted by methoxy or CF₃, and the aryl is optionally substituted by halogen; or

Het-(CH₂)ᵣ—, wherein r is 0 and Het is saturated or unsaturated 5 to 7-membered heterocycle that is optionally benzo-fused, wherein the heterocycle portion is optionally substituted by C₁-C₄-alkyl, halogen, C₁-C₄-alkyloxy, halophenyl or halobenzylmercapto, and wherein the benzo portion is optionally substituted by halogen or methoxy.

An additionally preferred method for inhibiting pancreatic lipase is administering a compound of formula 1 wherein:

R⁴ is R⁷-A-NR⁶—;

R⁶ is hydrogen;

A is —O—C(O)—; and

R⁷ is C₁-C₁₈-alkyl, substituted by CF₃ or phenyl;

C₆-C₁₀-aryl;

C₆-C₁₀-aryl-C₁-C₄-alkyl, wherein the aryl and alkyl are each independently optionally substituted by C₁-C₄-alkyl, halogen, CF₃ or OCF₃, benzyloxy or phenyl; or

Het-(CH₂)ᵣ—, wherein r is 0 or 1 and Het is saturated or unsaturated 5 to 7-membered heterocycle that is optionally benzo-fused, and wherein the heterocycle portion is optionally substituted by C₁-C₄-alkyl or benzyl.

An additionally preferred method for inhibiting pancreatic lipase is administering a compound of formula 1 wherein:

R⁴ is R⁷-A-NR⁶—;

R⁶ is hydrogen;

A is —SO₂—; and

R⁷ is C₁-C₆-alkyl, optionally substituted by CF₃;

C₂-C₄-alkenyl, optionally substituted by phenyl;

C₆-C₁₀-aryl, optionally substituted by C₁-C₆-alkyl, halogen, C₁-C₄-alkyloxy or benzyl;

biphenylyl-C₁-C₄-alkyl, wherein the phenyl and alkyl are optionally substituted by halogen; or

Het-(CH₂)ᵣ—, wherein r is 0 and Het is saturated or unsaturated 5 to 7-membered heterocycle.

An additionally preferred method for inhibiting pancreatic lipase is administering a compound of formula 1 wherein:

R⁴ is R⁷-A-NR⁶—;

R⁶ is hydrogen;

A is —NHCO—; and

R⁷ is C₁-C₁₀-alkyl, optionally substituted by:

C₁-C₄-alkyloxycarbonyl;

(C₁-C₄-alkyl)₂N—; or

phenyl, optionally substituted by halogen or aminosulfonyl;

C₆-C₁₀-aryl, optionally substituted by:

C₁-C₆-alkyl, C₁-C₆-alkyloxy, C₁-C₆-alkyloxycarbonyl, wherein the

alkyl is optionally substituted by C₁-C₄-alkyloxycarbonyl or

carboxyl;

phenoxy;

OCF₃;

benzyl; or

pyridyl;

C₅-C₈-cycloalkyl, optionally substituted by hydroxy;

indanyl; or

Het-(CH₂)ᵣ—, wherein r is 0 or 1 and Het is saturated or unsaturated 5 to 7-membered heterocycle, optionally substituted by benzyl.

A further preferred method for inhibiting pancreatic lipase is administering a compound of formula 1 wherein:

R² is hydrogen;

R⁵ is hydrogen;

R³ is hydrogen;

C₆-C₁₀-aryl;

C₆-C₁₀-aryloxy;

optionally C₁-C₄-alkyl-substituted C₆-C₁₀-aryloxymethyl; benzyloxy;

mono- or poly-fluorine- or amino-substituted C₁-C₆-alkyloxy, wherein the amino group is optionally substituted once or twice by times by C₁-C₄-alkyl; or

optionally mono- or poly-C₁-C₄-alkyl-substituted C₃-C₈-cycloalkyloxy; and

R⁴ is hydrogen;

C₆-C₁₀-aryl;

C₃-C₈-cycloalkyl;

optionally mono- or poly-C₁-C₄-alkyl- or halogen-substituted C₆-C₁₀ aryloxy or C₃-C₈-cycloalkyloxy;

mono- or poly-fluorine-substituted C₁-C₆-alkyloxy;

C₁-C₆-alkyl-NH—SO₂—, wherein the alkyl is optionally substituted by (C₁-C₆-alkyl)₂N—;

(2,2,6,6-tetramethylpiperidin-4-yl)-NH—SO₂—;

C₃-C₈-cycloalkyl-NH—SO₂—, wherein the cycloalkyl is optionally substituted one or more times by C₁-C₄-alkyl;

(C₁-C₆-alkyl)₂N—SO₂—;

YSO₂—, wherein Y is 1-piperidinyl, 4-morpholinyl or 1-piperazinyl, wherein the piperidinyl, morpholinyl and piperazinyl are each independently optionally substituted by C₁-C₄-alkyl; or

XCO—, wherein X is (C₁-C₆-alkyl)₂N—, 1-piperidinyl, 4-morpholinyl or 1-piperazinyl, wherein the piperidinyl, morpholinyl and piperazinyl are each independently optionally substituted by C₁-C₄-alkyl.

One particular preferred method for inhibiting pancreatic lipase is administering a compound of formula 1 wherein:

R¹ is methyl, ethyl, butyl, isopropyl or benzyl;

R² and R⁵ are hydrogen;

R³ is hydrogen, OCF₃, trifluorobutoxy, 3,3,5,5-tetramethylcyclohexyloxy, benzyloxy, phenoxy, phenyl, 2-diethylamino-ethyloxy or 3-methylphenoxymethyl; and

R⁴ is hydrogen, OCF₃, 3,3,5,5-tetramethylcyclohexyloxy, phenoxy, 4-chlorophenoxy, cyclohexyl, phenyl, morpholinosulfonyl, 3,3,5-trimethylcyclohexylaminosulfonyl, 2,2,6,6-tetramethylpiperid in-4-ylaminosulfonyl, 2-(diisopropylaminoethyl)aminosulfonyl, 4-methylpiperazin-1-ylsulfonyl, 3,3-dimethylpiperidinocarbonyl or 3,5-dichlorophenoxy.

Another particular preferred method for inhibiting pancreatic lipase is administering a compound of formula 1 wherein:

R¹ is methyl, ethyl, butyl, isopropyl or benzyl;

R² and R⁵ are hydrogen;

R³ is hydrogen, OCF₃, 3,3,5,5-tetramethylcyclohexyloxy, benzyloxy or phenoxy; and

R⁴ is hydrogen, OCF₃, 3,3,5,5-tetramethylcyclohexyloxy, phenoxy, cyclohexyl, phenyl, morpholinosulfonyl or 3,3,5-trimethylcyclohexyl-aminosulfonyl.

The very particularly preferred method for inhibiting pancreatic lipase is administering a compound of formula 1 wherein:

R¹ is C₁-C₄-alkyl;

R² is hydrogen;

R³ is hydrogen, OCF₃, benzyloxy;

R⁴ is hydrogen, OCF₃, 4-chlorophenoxy, 4-trifluoromethylbenzoylamino; and

R⁵ is hydrogen.

A further very particularly preferred method for inhibiting pancreatic lipase is administering a compound of formula 1 wherein: R¹ is methyl.

An additional very particularly preferred method for inhibiting pancreatic lipase is administering a compound of formula 1, which is:

5-Methoxy-3-(3-benzyloxy-4-(4-trifluoromethyl benzoylamino)phenyl)-3H-(1,3,4)oxadiazol-2-one;

3-(4-Trifluoromethoxyphenyl)-5-ethoxy-3H-(1,3,4)-oxad iazol-2-one;

3-(4-Trifluoromethoxyphenyl)-5-butoxy-3H-(1,3,4)-oxad iazol-2-one;

3-(4-Trifluoromethoxyphenyl)-5-benzyloxy-3H-(1,3,4)-oxadiazol-2-one;

3-(3-Benzyloxyphenyl)-5-methoxy-3H-(1,3,4)-oxad iazol-2-one;

3-(3-Trifluoromethoxyphenyl)-5-ethoxy-3H-(1,3,4)-oxadiazol-2-one;

3-(3-Trifluoromethoxyphenyl)-5-isopropoxy-3H-(1,3,4)-oxadiazol-2-one; or

3-(4-(4-Chlorophenoxy)phenyl)-5-methoxy-3H-(1,3,4)-oxad iazol-2-one.

The invention also encompasses all combinations of particular or preferred aspects of the invention noted herein.

It will be apparent to those skilled in the art that certain compounds of formula 1 can exhibit isomerism, for example geometrical isomerism, e.g., E or Z isomerism, and optical isomerism, e.g., R or S configurations. Geometrical isomers include the cis and trans forms of compounds of the invention having alkenyl moieties. Individual geometrical isomers and stereoisomers, including enantiomers and diastereoisomers, within formula 1, and their mixtures, are within the scope of the invention.

Pharmaceutically acceptable salts are particularly suitable for medical applications because of their greater solubility in water compared with the initial compounds on which they are based. These salts must have a pharmaceutically acceptable anion or cation. Suitable pharmaceutically acceptable acid addition salts of the compounds of formula I are salts of inorganic acids such as hydrochloric acid, hydrobromic acid, phosphoric, metaphosphoric, nitric, sulfonic and sulfuric acids, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic, tartaric and trifluoroacetic acids. It is particularly preferred to use the chloride salt and the tartaric acid salt for medical purposes. Suitable pharmaceutically acceptable basic salts are ammonium salts, alkali metal salts (such as sodium and potassium salts) and alkaline earth metal salts (such as magnesium and calcium salts). Salts with other anions such as perchlorate, hypochorite, tetrafluoroborate, hexachloroantimonate, picrate and azide, likewise fall within the scope of the invention as useful intermediates for preparing or purifying pharmaceutically acceptable salts and/or for use in non-therapeutic, for example in vitro, applications.

The term “physiologically functional derivative (prodrug)” used herein refers to any physiologically tolerated derivative of a compound according to the invention, for example an ester that is able on administration to a mammal, such as, for example, to humans, to form (directly or indirectly) such a compound or an active metabolite thereof. Such prodrugs can be metabolized in vivo to a compound of the formula 1. These prodrugs may themselves be active or not.

The compounds of formula 1 may also exist in various polymorphous forms, for example as amorphous and crystalline polymorphous forms. All polymorphous forms of the compounds of formula 1 fall within the scope of the invention and are a further aspect of the invention.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include ethanolates, methanolates, and the like.

The amount of a compound of formula 1 necessary to effect the method according to the invention, i.e., to achieve the desired biological effect depends on a number of factors, for example the specific compound chosen, the intended use, the mode of administration and the clinical condition of the patient. The daily dose is generally in the range from 0.3 mg to 100 mg (typically from 3 mg to 50 mg) per day and per kilogram of body weight, for example 3-10 mg/kg/day. An intravenous dose may be, for example, in the range from 0.3 mg to 1.0 mg/kg, which can suitably be administered as infusion of 10 ng to 100 ng per kilogram and per minute. Infusion solutions suitable for these purposes may contain, for example, from 0.1 ng to 10 mg, typically from 1 ng to 10 mg, per milliliter. Single doses may contain, for example, from 1 mg to 10 g of the active ingredient. Thus, ampoules for injections may contain, for example, from 1 mg to 100 mg, and single dose formulations that can be administered orally, such as, for example, tablets or capsules, may contain, for example, from 1.0 to 1000 mg, typically from 10 to 600 mg. In the case of pharmaceutically acceptable salts, the above weight data are based on the weight of the salt of the compound of formula 1. The compounds of formula 1 can be used for prophylaxis or therapy of the abovementioned states themselves as compound, but they are preferably in the form of a pharmaceutical composition with a compatible carrier. The carrier must, of course, be compatible in the sense of compatibility with other ingredients of the composition and not be harmful to the patient's health. The carrier may be a solid or a liquid or both and is preferably formulated with the compound as single dose, for example as tablet, which may contain from 0.05% to 95% by weight of the active ingredient. Further pharmaceutically active substances may likewise be present, including further compounds of formula 1. The pharmaceutical compositions according to the invention may be produced by one of the known pharmaceutical methods that essentially consist of mixing the ingredients with pharmacologically acceptable carriers and/or excipients.

Pharmaceutical compositions according to the invention are those suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the nature of the compound of formula 1 used in each case. Coated formulations and coated slow-release formulations also fall within the scope of the invention. Acid- and gastric fluid-resistant formulations are preferred. Suitable gastric fluid-resistant coatings comprise cellulose acetate phthalate, polyvinyl acetate phthalate, hydroxypropylmethyl-cellulose phthalate and anionic polymers of methacrylic acid and methyl methacrylate.

Suitable pharmaceutical compounds for oral administration may be in the form of separate units such as, for example, capsules, cachets, pastilles or tablets, each of which contains a defined amount of the compound of formula 1; as powder or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. In general, the compositions are produced by uniform and homogeneous mixing of the active ingredient with a liquid and/or finely dispersed solid carrier, after which the product is shaped if necessary. Thus, for example, a tablet can be produced by compressing or shaping a powder or granules of the compound, where appropriate with one or more additional ingredients. Compressed tablets may be produced by tabletting the compound in free-flowing form, such as, for example, a powder or granules, where appropriate mixed with a binder, lubricant, inert diluent and/or one (or more) surface-active/dispersing agents in a suitable machine. Shaped tablets can be produced by shaping, in a suitable machine, the compound that is in powder form and has been moistened with an inert liquid diluent.

Pharmaceutical compositions suitable for peroral (sublingual) administration comprise suckable tablets that contain a compound of formula 1 with a flavoring, normally sucrose, and gum arabic or tragacanth, and pastilles that contain the compound in an inert base such as gelatin and glycerol or sucrose and gum arabic.

Suitable pharmaceutical compositions for parenteral administration comprise preferably sterile aqueous preparations of a compound of formula 1, which are preferably isotonic with the blood of the intended recipient. These preparations are preferably administered intravenously, although administration can also take place by subcutaneous, intramuscular or intradermal injection. These preparations can preferably be produced by mixing the compound with water and making the resulting solution sterile and isotonic with blood. Injectable compositions according to the invention generally contain from 0.1 to 5% by weight of the active compound.

Suitable pharmaceutical compositions for rectal administration are preferably in the form of single-dose suppositories. These can be produced by mixing a compound of formula 1 with one or more conventional solid carriers, for example cocoa butter, and shaping the resulting mixture.

Suitable pharmaceutical compositions for topical use on the skin are preferably in the form of an ointment, cream, lotion, paste, spray, aerosol or oil. Carriers that can be used are petrolatum, lanolin, polyethylene glycols, alcohols and combinations of two or more of these substances. The active ingredient is generally present in a concentration of from 0.1 to 15% by weight of the composition, for example from 0.5 to 2%.

Transdermal administration is also possible. Suitable pharmaceutical compositions for transdermal applications may be in the form of single plasters that are suitable for long-term close contact with the patient's epidermis. Plasters of this type suitably contain the active ingredient in an aqueous solution that is buffered where appropriate, dissolved and/or dispersed in an adhesive or dispersed in a polymer. A suitable active ingredient concentration is about 1% to 35%, preferably about 3% to 15%. As a particular option, the active ingredient can be released by electrotransport or iontophoresis as described, for example, in Pharmaceutical Research, 2 (6): 318 (1986).

The following preparations serve to illustrate the invention without, however, restricting it.

### EXAMPLE A

Soft gelatin capsules containing 100 mg of active ingredient per capsule:

| | per capsule |
|---|---|
| active ingredient | 100 mg |
| triglyceride mixture | 400 mg |
| fractionated from coconut fat | |
| capsule content | 500 mg |

### EXAMPLE B

Emulsion containing 60 mg of active ingredient per 5 mL:

| | per 100 mL of emulsion | |
|---|---|---|
| active ingredient | 1.2 | g |
| neutral oil | q.s. | |
| sodium carboxymethylcellulose | 0.6 | g |
| polyoxyethylene stearate | q.s. | |
| glycerol, pure | 0.2 | to |
| | 2.0 | g |
| flavoring | q.s. | |
| water (deionized or distilled) | ad 100 | mL |

### EXAMPLE C

Rectal drug form containing 40 mg of active ingredient per suppository:

| | per suppository | |
|---|---|---|
| active ingredient | 40 | mg |
| suppository base | ad 2 | g |

### EXAMPLE D

Tablets containing 40 mg of active ingredient per tablet:

| | per tablet | |
|---|---|---|
| active ingredient | 40 | mg |
| lactose | 600 | mg |
| corn starch | 300 | mg |
| soluble starch | 20 | mg |
| magnesium stearate | 40 | mg |
| | 1000 | mg |

### EXAMPLE E

Coated tablets containing 50 mg of active ingredient per tablet:

| | per tablet | |
|---|---|---|
| active ingredient | 50 | mg |
| corn starch | 100 | mg |
| lactose | 60 | mg |
| sec. calcium phosphate | 30 | mg |
| soluble starch | 5 | mg |
| magnesium stearate | 10 | mg |
| colloidal silica | 5 | mg |
| | 260 | mg |

### EXAMPLE F

The following formulas are suitable for producing the contents of hard gelatin capsules:

| | | |
|---|---|---|
| a) | active ingredient | 100 mg |
| | corn starch | 300 mg |
| | | 400 mg |
| b) | active ingredient | 140 mg |
| | lactose | 180 mg |
| | corn starch | 180 mg |
| | | 500 mg |

### EXAMPLE G

Drops can be produced in accordance with the following formula (100 mg of active ingredient in 1 mL=20 drops):

| | | |
|---|---|---|
| active ingredient | 10 | g |
| methyl benzoate | 0.07 | g |
| ethyl benzoate | 0.03 | g |
| ethanol, 96% | 5 | mL |
| demineralized water | ad 100 | mL |

The compounds of formula 1 can be prepared in various ways by methods known per se.

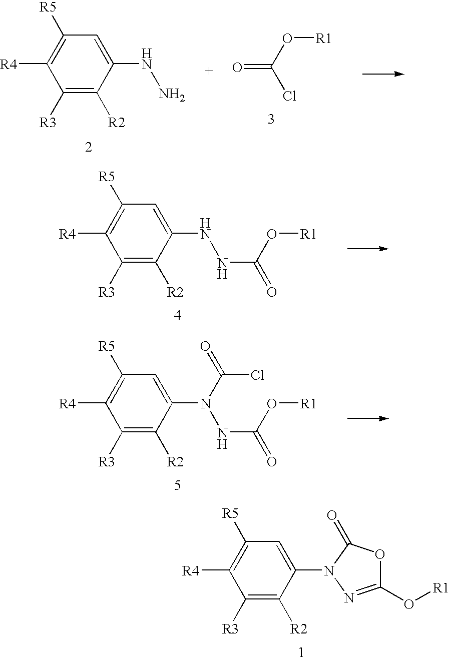

For example, substituted 3-phenyl-5-alkoxy-3H-(1,3,4)-oxadiazol-2-one of formula 1 can be prepared by reacting a hydrazine of formula 2 with a chloroformic ester of formula 3 or other reactive carbonic ester derivative, wherein R¹, R², R³, R⁴ and R⁵ are as defined above, to give a compound of formula 4, which is acylated with phosgene (for example to give a compound of formula 5), carbonyldiimidazole, diphosgene or triphosgene, cyclized and converted where appropriate by further chemical modification of the radicals R²-R⁵, such as, for example, by reduction of nitro to amino radicals by known processes, and subsequent acylation or alkylation, into compounds of formula 1. Since acids are usually liberated in these reactions, promotion is advisable by adding bases such as pyridine, triethylamine, sodium hydroxide solution or alkali metal carbonates. The reactions can be carried out in wide temperature ranges. It has proved advantageous as a rule to operate at 0° C. to the boiling point of the solvent used. Examples of solvents employed are methylene chloride, THF, DMF, toluene, ethyl acetate, n-heptane, dioxane, diethyl ether.

The hydrazines of formula 2 can be prepared by known methods, for example by diazotization of the corresponding anilines and

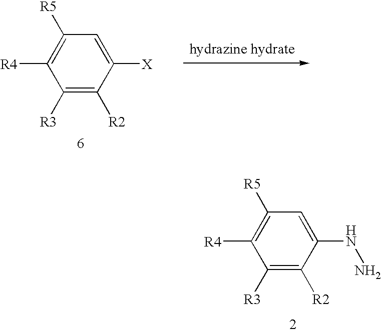

subsequent reduction by known methods or by nucleophilic substitution of suitably substituted phenyl derivatives 6 (X=F, Cl, Br, I, OSO₂CF₃) with hydrazine hydrate. Such suitable phenyl derivatives may be nitro-substituted halobenzenes, preferably fluoro- and chloronitrobenzenes, from which the compounds of formula 1 can be prepared by known methods at a suitable point in the synthetic route by reduction and reaction with acylating or alkylating agents such as, for example, acid chlorides, anhydrides, isocyanates, chloroformic esters, sulfonyl chlorides or alkyl and arylalkyl halides, or by reductive alkylation with aldehydes.

The following examples illustrate the preparation methods in detail without restricting them.

### EXAMPLES

### Example 1

3-Methyl-4-nitrophenylhydrazine

5 g of hydrazine hydrate are slowly added dropwise to a solution of 15.9 g of 2-methyl-4-fluoronitrobenzene in 10 mL of N-methylpyrrolidone at room temperature, and the mixture is heated with stirring at 65° C. for 4 hours. The product is precipitated by adding 70 mL of water and is filtered off with suction and recrystallized from isopropanol.

Yield:13.3 g, m.p.: 138° C.

The following examples were prepared in an analogous way:

### Example 2

3-Fluoro-4-nitrophenylhydrazine

m.p.: 130° C.

### Example 3

2-Chloro-4-nitrophenylhydrazine

m.p.:144° C.

### Example 4

2-Methyl-4-nitrophenylhydrazine

m.p.:135° C.

### Example 5

3-(4-Fluorobenzyloxy)-2-nitrophenylhydrazine

m.p.:164° C.

The starting compound 2-fluoro-4-(4-fluorobenzyloxy)nitrobenzene (m.p.: 99° C.) was prepared by alkylation of 3-fluoro-4-nitrophenol with 4-fluorobenzyl chloride in DMF in the presence of potassium carbonate.

### Example 6

3-(4-Fluorobenzyloxy)-4-nitrophenylhydrazine (intermediate)

m.p.: 145° C.

### Example 7

4-(4-Chlorophenoxy)-3-nitroaniline

1.4 g of potassium carbonate are added to a solution of 1.29 g of 4-chlorophenol in 8 mL of DMF and, after stirring for 30 minutes, 1.6 g of 4-fluoro-3-nitroaniline are added, and the mixture is stirred at 100° C. for 3 hours. After cooling, 80 mL of water are added and, after briefly stirring, the precipitate is filtered off with suction and dried in vacuo at 40° C.

Yield: 2.0 g; m.p.: 101° C.

### Example 8

4-(4-Chlorophenoxy)-3-nitrophenylhydrazine

A solution of 0.52 g of sodium nitrite in 5 mL of water is added dropwise to a stirred mixture consisting of 1.9 g of 4-(4-chlorophenoxy)-3-nitroaniline, 25 mL of concentrated hydrochloric acid and 25 mL of ethanol cooled to 0° C., and the mixture is then stirred at 0° C. for 60 min and subsequently added dropwise to a suspension of 8.5 g of tin dichloride dihydrate in 8 mL of concentrated HCl. The precipitate is filtered off with suction, washed with water, suspended in 200 mL of water under nitrogen and decomposed with 100 mL of 30% strength sodium hydroxide solution at 10-15° C. The oil that forms is extracted by shaking with ethyl acetate and washed with water, and the organic phase is dried with sodium sulfate. The product is then precipitated with isopropanolic HCl, filtered off with suction and dried in vacuo.

Yield: 1.1 g; m.p.: 221° C.

### Example 9

Methyl N′-(4-nitro-2-methyl phenyl)hydrazinoformate

0.43 mL of methyl chloroformate was cautiously added dropwise to a mixture consisting of 0.84 g of 2-methyl-4-nitrophenylhydrazine, 15 mL of N-methyl pyrrolidinone and 2 mL of pyridine while cooling in ice, and the mixture was then stirred for 2 hours while slowly warming to room temperature. After dilution with 50 mL of water, the mixture was stirred overnight and the solid was dried in vacuo at 40° C.

Yield: 0.81 g; m.p.:153° C.

The following examples were prepared in an analogous way:

### Example 10

Methyl N′-(4-nitrophenyl)hydrazinoformate (intermediate)

m.p.: 179° C.

### Example 11

Methyl N′-(3-fluoro-4-nitrophenyl)hydrazinoformate

m.p.: 127.4° C.

### Example 12

Methyl N′-(3-methyl-4-nitrophenyl)hydrazinoformate

m.p.: 159° C.

### Example 13

Methyl N′-(2-chloro-4-nitrophenyl)hydrazinoformate

m.p.: 156° C.

### Example 14

Methyl N′-(3-(4-fluorobenzyloxy)-4-nitrophenyl)hydrazinoformate (intermediate)

m.p.: 166° C.

### Example 15

Methyl N′-(3-(4-fluorobenzyloxy)-2-nitrophenyl)hydrazinoformate

m.p.: 193° C.

### Example 16

Methyl N′-(4-(4-chlorophenoxy)-3-nitrophenyl)hydrazinoformate

m.p.: 147° C.

### Example 17

Methyl N′-(3-piperidino-4-nitrophenyl)hydrazinoformate (−)

m.p.: 131° C.

The latter compound and the compound of Example 18 were prepared by reacting methyl N′-(3-fluoro-4-nitrophenyl)hydrazinoformate with piperidine and N-benzyl-piperazine, respectively, in NMP at 80° C.

### Example 18

Methyl N′-(3-(N-benzylpiperazino)-4-nitrophenyl)hydrazinoformate

m.p.: 156° C.

### Example 19

5-Methoxy-3-(4-nitrophenyl)-3H-(1,3,4)oxadiazol-2-one 2.5 g of methyl N′-(4-nitrophenyl)hydrazinoformate and 5 mL of pyridine were taken up in 15 mL of methylene chloride and, while stirring and cooling in ice, 3 mL of a 20% strength solution of phosgene in toluene were added dropwise. This mixture was left to stand at room temperature overnight and was diluted with a further 10 mL of methylene chloride and then washed 3 times with water. After drying over sodium sulfate, the mixture was concentrated in vacuo, and the product was purified by column chromatography (silica gel, solvents: methanol:methylene chloride=2:98) and recrystallized from isopropanol.

Yield:1.5 g m.p.: 151° C.

The following examples were prepared in analogy to Example 4:

### Example 20

5-Methoxy-3-(3-methyl-4-nitrophenyl)-3H-(1,3,4)-oxad iazol-2-one

m.p.: 112° C.

### Example 21

5-Methoxy-3-(4-(4-chlorophenoxy-3-nitrophenyl)-3H-(1,3,4)-oxadiazol-2-one

m.p.: oil

### Example 22

5-Methoxy-3-(3-(4-fluorobenzyloxy)-2-nitrophenyl)-3H-(1,3,4)-oxadiazol-2-one

m.p.: 99° C.

### Example 23

5-Methoxy-3-(2-methyl-4-nitrophenyl)-3H-(1,3,4)-oxad iazol-2-one

m.p.: 111° C.

### Example 24

5-Methoxy-3-(3-(4-fluorobenzyloxy)-4-nitrophenyl)-3H-(1,3,4)-oxadiazol-2-one

m.p.: 137° C.

### Example 25

5-Methoxy-3-(4-aminophenyl)-3H-(1,3,4)-oxad iazol-2-one

A mixture consisting of 1.4 g of 5-methoxy-3-(4-nitrophenyl)-3H-(1,3,4)-oxadiazol-2-one, 0.5 g of Pd/C and 20 mL of methanol is hydrogenated under atmospheric pressure at room temperature until the calculated amount of hydrogen has been taken up. The catalyst is then filtered off, and the solution is concentrated in vacuo. The remaining semisolid residue is stirred with isopropanol and filtered off with suction.

Yield: 0.75 g; m.p.: 85° C.

### Example 26

5-Methoxy-3-(2-amino-4-(4-fluorobenzyloxy)phenyl)-3H-(1,3,4)-oxad iazol-2-one

m.p.: oil

### Example 27

5-Methoxy-3-(3-amino-4-(4-chlorophenoxy)phenyl)-3H-(1,3,4)-oxadiazol-2-one

m.p.: 133° C.

### Example 28

5-Methoxy-3-(4-amino-3-methyl phenyl)-3H-(1,3,4)-oxad iazol-2-one

m.p.: 114° C.

### Example 29

5-Methoxy-3-(4-amino-3-(4-fluorobenzyloxy)phenyl)-3H-(1,3,4)-oxadiazol-2-one

m.p.: 195° C.

### Example 30

5-Methoxy-3-(4-(4-chlorophenylacetylamino)phenyl)-3H-(1,3,4)-oxadiazol-2-one

201 mg of 4-chlorophenylacetyl chloride are added dropwise to a mixture consisting of 200 mg of 5-methoxy-3-(4-aminophenyl)-3H-(1,3,4)-oxadiazol-2-one, 20 mL of methylene chloride and 0.1 mL of pyridine cooled in ice, and the mixture is stirred at room temperature for 5 hours. Volatiles are removed in vacuo, and the residue is stirred with water and the solid is filtered off with suction and dried at 40° C. in vacuo.

Yield: 318 mg; m.p.:161° C.

The following examples were prepared in an analogous way:

### Example 31

5-Methoxy-3-(4-(4-chlorophenylacetylamino)-3-methylphenyl)-3H-(1,3,4)-oxadiazol-2-one

m.p.: 190° C.

### Example 32

5-Methoxy-3-(4-octanoylamino-3-methylphenyl)-3H-(1,3,4)-oxad iazol-2-one

m.p.: 110° C.

### Example 33

5-Methoxy-3-(4-(4-heptylbenzoylamino)phenyl)-3H-(1,3,4)-oxadiazol-2-one

m.p.: 155° C.

### Example 34

5-Methoxy-3-(4-(4-butylphenylsulfonylamino)phenyl)-3H-(1,3,4)-oxad iazol-2-one

m.p.: 1.35° C.

### Example 35

5-Methoxy-3-(4-(4-chlorobutanoylamino)-3-methylphenyl)-3H-(1,3,4)-oxadiazol-2-one

m.p.: 137° C.

### Example 36

5-Methoxy-3-(4-pivaloylamino-3-methylphenyl)-3H-(1,3,4)-oxad iazol-2-one

m.p.: 157° C.

### Example 37

5-Methoxy-3-(4-(4-chlorophenylsulfonylamino)-3-methylphenyl)-3H-(1,3,4)-oxadiazol-2-one

m.p.: 147° C.

### Example 38

5-Methoxy-3-(4-(1-naphthylsulfonylamino)-3-methylphenyl)-3H-(1,3,4)-oxadiazol-2-one

m.p.: 123° C.

### Example 39

5-Methoxy-3-(4-(2-phenylethenylsulfonylamino)-3-methylphenyl)-3H-(1,3,4)-oxadiazol-2-one

m.p.: 129° C.

### Example 40

5-Methoxy-3-(4-(2,2,2-trifluoroethylsulfonylamino)-3-methylphenyl)-3H-(1,3,4)-oxadiazol-2-one

m.p.: 151° C.

### Example 41

5-Methoxy-3-(4-(benzyloxycarbonylamino)-3-methylphenyl)-3H-(1,3,4)-oxadiazol-2-one

m.p.: 115° C.

### Example 42

5-Methoxy-3-(4-(3,4-dichlorophenylaminocarbonylamino)-3-methylphenyl)-3H-(1,3,4)-oxadiazol-2-one

m.p.: 210° C.

The latter compound was obtained by reacting 5-methoxy-3-(4-amino-3-methylphenyl)-3H-(1,3,4)-oxadiazol-2-one with equimolar amounts of 3,4-dichlorophenyl isocyanate in toluene at 50° C.

### Example 43

5-Methoxy-3-(4-(4-chlorophenylsulfonylamino)phenyl)-3H-(1,3,4)-oxadiazol-2-one

m.p.: 169° C.

### Example 44

5-Methoxy-3-(4-(2-chlorophenylsulfonylamino)phenyl)-3H-(1,3,4)-oxadiazol-2-one

m.p.: 171° C.

### Example 45

5-Methoxy-3-(4-(3-chlorophenylsulfonylamino)phenyl)-3H-(1,3,4)-oxadiazol-2-one

m.p.: 141° C.

### Example 46

5-Methoxy-3-(4-(4-chlorobenzoylamino)-3-(4-fluorobenzyloxy)phenyl)-3H-(1,3,4)-oxadiazol-2-one

m.p.: 167° C.

### Example 47

5-Methoxy-3-(4-benzylsulfonylaminophenyl)-3H-(1,3,4)-oxad iazol-2-one

m.p.: 153° C.

### Example 48

5-Methoxy-3-(4-(-2-(4′-chlorobiphenyl)ethyl)sulfonylamino)phenyl)-3H-(1,3,4)-oxadiazol-2-one

m.p.: 165° C.

### Example 49

5-Methoxy-3-(4-isopropylsulfonylaminophenyl)-3H-(1,3,4)-oxadiazol-2-one

m.p.: 190° C.

### Example 50

5-Methoxy-3-(4-dimethylamino-3-methylphenyl)-3H-(1,3,4)-oxadiazol-2-one

m.p.: 71° C.

The latter compound was obtained by reacting 5-methoxy-3-(4-amino-3-methylphenyl)-3H-(1,3,4)-oxadiazol-2-one with paraformaldehyde/formic acid in DMF at room temperature and was purified by column chromatography (silica gel, ethyl acetate:n-heptane=1:1).

### Example 51

5-Methoxy-3-(4-(4-chlorobenzylamino)-3-methylphenyl)-3H-(1,3,4)-oxadiazol-2-one

m.p.: oil

The latter compound was obtained by reacting 5-methoxy-3-(4-amino-3-methylphenyl)-3H-(1,3,4)-oxadiazol-2-one with 4-chlorobenzaldehyde/sodium borohydride in methanol/methylene chloride at room temperature and was purified by column chromatography (silica gel, ethyl acetate:n-heptane=1:1).

### Example 52

5-Methoxy-3-(4-(2-oxopyrrolidin-1-yl)-3-methylphenyl)-3H-(1,3,4)-oxad iazol-2-one

m.p.: oil

The latter compound was prepared by reacting 5-methoxy-3-(4-(4-chlorobutanoylamino)-3-methylphenyl)-3H-(1,3,4)-oxad iazol-2-one with sodium hydride in dioxane at room temperature and purifying the crude product by column chromatography (siilca gel, methylene chloride:methanol=98:2).

### Example 53

5-Methoxy-3-(4-(4-oxopent-2-en-2-ylamino)-3-methylphenyl)-3H-(1,3,4)oxad iazol-2-one

m.p.: 143° C.

The latter compound was obtained by reacting 5-methoxy-3-(4-amino-3-methylphenyl)-3H-(1,3,4)-oxadiazol-2-one with equimolar amounts of acetylacetone in glacial acetic acid at 80° C. and was isolated by precipitation by adding water and filtration.

### Example 54

5-Methoxy-3-(4-(2,5-dimethylpyrrol-1-yl)-3-methylphenyl)-3H-(1,3,4)-oxad iazol-2-one

m.p.: oil

The latter compound was obtained by reacting 5-methoxy-3-(4-amino-3-methylphenyl)-3H-(1,3,4)-oxadiazol-2-one with equimolar amounts of acetonylacetone in glacial acetic acid at 80° C. Working up took place by dilution with water, extraction by shaking with ethyl acetate and column chromatography (silica gel, methylene chloride) of the crude product obtained after concentration of the dried organic phase.

### Example 55

5-Methoxy-3-(3-(4-fluorobenzyloxy)-4-methylaminophenyl)-3H-(1,3,4)-oxad iazol-2-one

m.p.: 98° C.

The latter compound was obtained as by-product of the hydrogenation of 5-methoxy-3-(3-(4-fluorobenzyloxy)-4-nitrophenyl)-3H-(1,3,4)-oxadiazol-2-one with platinum dioxide as catalyst in methanol at room temperature under atmospheric pressure and after filtering off the catalyst, concentrating the reaction mixture and column chromatography (silica gel, methylene chloride).

The compounds of Examples 56-199 were prepared analogously to the above examples.

### Example 56

5-Methoxy-3-(3-aminophenyl)-3H-(1,3,4)-oxad iazol-2-one

m.p.: 95° C.

### Example 57

5-Methoxy-3-(3-dibenzylaminophenyl)-3H-(1,3,4)-oxadiazol-2-one

m.p.: 71° C.

### Example 58

5-Methoxy-3-(3-benzylaminophenyl)-3H-(1,3,4)-oxadiazol-2-one

m.p.: oil

### Example 59

5-Methoxy-3-(3-(pyrid-2-yl)aminocarbonylaminophenyl)-3H-(1,3,4)-oxadiazol-2-one

m.p.: 81° C.

### Example 60

5-Methoxy-3-(3-(4-fluorobenzyloxy)-4-benzyloxycarbonylaminophenyl)-3H-(1,3,4)-oxadiazol-2-one

m.p.: oil

### Example 61

5-Methoxy-3-(4-amino-2-methylphenyl)-3H-(1,3,4)-oxadiazol-2-one

m.p.: oil

### Example 62

5-Methoxy-3-(3-methyl-4-(2-chlorobenzyloxycarbonylamino)phenyl)-3H-(1,3,4)-oxadiazol-2-one

m.p.: 161° C.

### Example 63

5-Methoxy-3-(4-amino-2-chlorophenyl)-3H-(1,3,4)-oxadiazol-2-one

m.p.: 126° C.

### Example 64

5-Methoxy-3-(2-chloro-4-nitrophenyl)-3H-(1,3,4)-oxad iazol-2-one

m.p.: 92° C.

### Example 65

5-Methoxy-3-(2-methyl-4-benzyloxycarbonylaminophenyl)-3H-(1,3,4)-oxadiazol-2-one

m.p.: 112° C.

### Example 66

5-Methoxy-3-(2-methyl-4-(4-trifluoromethoxybenzoylamino)phenyl)-3H-(1,3,4)-oxadiazol-2-one

m.p.: 150° C.

### Example 67

5-Methoxy-3-(2-chloro-4-benzyloxycarbonylaminophenyl)-3H-(1,3,4)-oxad iazol-2-one

m.p.: 150° C.

### Example 68

5-Methoxy-3-(3-fluoro-4-nitrophenyl)-3H-(1,3,4)-oxad iazol-2-one

m.p.: 127° C.

### Example 69

5-Methoxy-3-(4-(4-t-butylbenzoylamino)phenyl)-3H-(1,3,4)-oxadiazol-2-one

m.p.: 173° C.

### Example 70

5-Methoxy-3-(4-(4-chlorobenzyloxycarbonylamino)phenyl)-3H-(1,3,4)-oxadiazol-2-one

m.p.: 177° C.

### Example 71

5-Methoxy-3-(2-chloro-4-(4-heptylbenzoylamino)phenyl)-3H-(1,3,4)-oxadiazol-2-one

m.p.: 135° C.

### Example 72

5-Methoxy-3-(4-(3,4-dichlorobenzoylamino)phenyl)-3H-(1,3,4)-oxad iazol-2-one

m.p.: 200° C.

### Example 73

5-Methoxy-3-(4-(2-(4-chlorophenoxy)-2-methylpropionylamino)phenyl)-3H-(1,3,4)-oxadiazol-2-one

m.p.: 153° C.

### Example 74

5-Ethoxy-3-(3-methyl-4-benzyloxycarbonylaminophenyl)-3H-(1,3,4)-oxadiazol-2-one

m.p.: 94° C.

### Example 75

5-Isopropoxy-3-(3-methyl-4-benzyloxycarbonylaminophenyl)-3H-(1,3,4)-oxad iazol-2-one

m.p.: 119° C.

### Example 76

5-Isopropoxy-3-(3-methyl-4-butyloxycarbonylaminophenyl)-3H-(1,3,4)-oxadiazol-2-one

m.p.: 114° C.

### Example 77

5-Isopropoxy-3-(3-methyl-4-(3-chlorophenylaminocarbonylamino)phenyl)-3H-(1,3,4)-oxadiazol-2-one

m.p.: 201° C.

### Example 78

5-tert-Butoxy-3-(3-methyl-4-benzyloxycarbonylaminophenyl)-3H-(1,3,4)-oxad iazol-2-one

m.p.: 113° C.

### Example 79

5-Methoxy-3-(3-methyl-4-phenoxycarbonylaminophenyl)-3H-(1,3,4)-oxad iazol-2-one

m.p.: 145° C.

### Example 80

5-Methoxy-3-(3-methyl-4-(pyrid-3-ylcarbonylamino)phenyl)-3H-(1,3,4)-oxad iazol-2-one

m.p.: oil

### Example 81

5-Methoxy-3-(3-methyl-4-(indan-2-ylaminocarbonylamino)phenyl)-3H-(1,3,4)-oxadiazol-2-one

m.p.: 206° C.

### Example 82

5-Methoxy-3-(3-methyl-4-(pyrid-3-ylmethylaminocarbonylamino)phenyl)-3H-(1,3,4)-oxadiazol-2-one

m.p.: 229° C.

### Example 83

5-Methoxy-3-(3-methyl-4-(pyrid-3-ylmethoxycarbonylamino)phenyl)-3H-(1,3,4)-oxadiazol-2-one

m.p.: 232° C.

### Example 84

5-Methoxy-3-(3-fluoro-4-benzyloxycarbonylaminophenyl)-3H-(1,3,4)oxadiazol-2-one

m.p.: oil

### Example 85

5-Methoxy-3-(3-fluoro-4-(4-trifluoromethylbenzoylamino)phenyl)-3H-(1,3,4)-oxadiazol-2-one

m.p.: oil

### Example 86

5-Methoxy-3-(3-benzyloxy-4-(4-trifluoromethyl benzoylamino)phenyl)-3H-(1,3,4)-oxadiazol-2-one

m.p.: 159° C.

### Example 87

5-Methoxy-3-(3-fluoro-4-(4-tert-butylbenzoylamino)phenyl)-3H-(1,3,4)-oxad iazol-2-one

m.p.: 144° C.

### Example 88

5-Methoxy-3-(3-methyl-4-(2,2,2-trifluoroethoxycarbonylamino)phenyl)-3H-(1,3,4)-oxadiazol-2-one

m.p.: 141° C.

### Example 89

5-Methoxy-3-(3-methyl-4-piperidinocarbonylaminophenyl)-3H-(1,3,4)-oxadiazo1-2-one

m.p.: 154° C.

Example 90

5-Methoxy-3-(4-(6-methoxybenzofuran-2-yl-carbonylamino)phenyl)-3H-(1,3,4)-oxadiazol-2-one

m.p.: 191° C.

Further examples which were prepared by the processes described above and were characterized by mass spectroscopy (M+1):

| Example | | | |
|---|---|---|---|
| No. | Chemical name: | M + 1 | Mol. wt. |
| 91 | N-[4-(5-Methoxy-2-oxo-[1,3,4]-oxadiazol-3-yl)phenyl]-3-methyl- | 362 | 361.4 |
| | benzenesulfonamide | | |
| 92 | 3,4-Dimethoxy-N-[4-(5-methoxy-2-oxo-[1,3,4]-oxadiazol-3-yl)- | 408 | 407.4 |
| | phenyl]benzenesulfonamide | | |
| 93 | Quinoline-8-sulfonic acid [4-(5-methoxy-2-oxo-[1,3,4]-oxadiazol- | 399 | 398.4 |
| | 3-yl)phenyl]amide | | |
| 94 | N-[4-(5-Methoxy-2-oxo-[1,3,4]-oxadiazol-3-yl)phenyl]-5-nitro- | 415 | 414.3 |
| | isophthalic acid monomethyl ester | | |
| 95 | 3-(2-Chlorophenyl)-5-methylisoxazole-4-carboxylic acid | 427 | 426.8 |
| | [4-(5-methoxy-2-oxo-[1,3,4]-oxadiazol-3-yl)phenyl]amide | | |
| 96 | 3,3,3-Trifluoro-2-methoxy-N-[4-(5-methoxy-2-oxo-[1,3,4]- | 424 | 423.3 |
| | oxadiazol-3-yl)phenyl]-2-phenylpropionamide | | |
| 97 | 2-Fluoro-N-[4-(5-methoxy-2-oxo-[1,3,4]-oxadiazol-3-yl)phenyl]- | 330 | 329.3 |
| | benzamide | | |
| 98 | Tetradecanoic acid [4-(5-methoxy-2-oxo-[1,3,4]-oxadiazol- | 418 | 417.5 |
| | 3-yl)phenyl]amide | | |
| 99 | N-[4-(5-Methoxy-2-oxo-[1,3,4]-oxadiazol-3-yl)phenyl]-2-phenethyl- | 416 | 415.4 |
| | benzamide | | |
| 100 | N-[4-(5-Methoxy-2-oxo-[1,3,4]-oxadiazol-3-yl)phenyl]-2-(4- | 479 | 478.4 |
| | methoxyphenoxy)-5-nitrobenzamide | | |
| 101 | 2-(4-Benzyloxyphenyl)-N-[4-(5-methoxy-2-oxo-[1,3,4]-oxadiazol- | 432 | 431.4 |
| | 3-yl)phenyl]acetamide | | |
| 102 | N-[4-(5-Methoxy-2-oxo-[1,3,4]-oxadiazol-3-yl)phenyl]- | 492 | 491.5 |
| | 3,3,3-triphenylpropionamide | | |
| 103 | N-[4-(5-Methoxy-2-oxo-[1,3,4]-oxadiazol-3-yl)phenyl]-3,5-bis- | 448 | 447.3 |
| | trifluoromethylbenzamide | | |
| 104 | 4-Cyano-N-[4-(5-methoxy-2-oxo-[1,3,4]-oxadiazol-3-yl)phenyl]- | 337 | 336.3 |
| | benzamide | | |
| 105 | Nonanoic acid [4-(5-methoxy-2-oxo-[1,3,4]-oxadiazol-3-yl)phenyl]- | 348 | 347.4 |
| | amide | | |
| 106 | Methyl 9-[4-(5-methoxy-2-oxo-[1,3,4]-oxadiazol-3-yl)phenyl- | 406 | 405.4 |
| | carbamoyl]nonanoate | | |
| 107 | Undecanoic acid [4-(5-methoxy-2-oxo-[1,3,4]-oxadiazol- | 376 | 375.5 |
| | 3-yl)phenyl]amide | | |
| 108 | 4-[4-(5-Methoxy-2-oxo-[1,3,4]-oxadiazol-3-yl)phenylcarbamoyl]- | 394 | 393.3 |
| | benzenesulfonyl fluoride | | |
| 109 | 11-Phenoxyundecanoic acid [4-(5-methoxy-2-oxo-[1,3,4]- | 468 | 467.6 |
| | oxadiazol-3-yl)phenyl]amide | | |
| 110 | N-[4-(5-Methoxy-2-oxo-[1,3,4]-oxadiazol-3-yl)phenyl]-2,3-diphenyl- | 416 | 415.4 |
| | propionamide | | |
| 111 | 4-Chloro-N-[4-(5-methoxy-2-oxo-[1,3,4]-oxadiazol-3-yl)phenyl]- | 360 | 359.8 |
| | 2-methylbenzamide | | |
| 112 | 6-Chloro-N-[4-(5-methoxy-2-oxo-[1,3,4]oxadiazol-3-yl)phenyl]- | 347 | 346.7 |
| | nicotinamide | | |
| 113 | 5-Fluoro-N-[4-(5-methoxy-2-oxo-[1,3,4]oxadiazol-3-yl)phenyl]- | 344 | 343.3 |
| | 2-methylbenzamide | | |
| 114 | N-[4-(5-Methoxy-2-oxo-[1,3,4]-oxadiazol-3-yl)phenyl]- | 354 | 353.4 |
| | 2,4,6-trimethylbenzamide | | |
| 115 | N-[4-(5-Methoxy-2-oxo-[1,3,4]-oxadiazol-3-yl)phenyl]-3- | 388 | 387.4 |
| | naphthalen-2-ylacrylamide | | |
| 116 | 5-Oxo-5-phenylpentanoic acid [4-(5-methoxy-2-oxo- | 382 | 381.4 |
| | [1,3,4]-oxadiazol-3-yl)phenyl]amide | | |
| 117 | 3-(2,4-Dichlorobenzylsulfanyl)thiophene-2-carboxylic acid | 509 | 508.4 |
| | [4-(5-methoxy-2-oxo-[1,3,4]-oxadiazol-3-yl)phenyl]amide | | |
| 118 | 2-Fluoro-N-[4-(5-methoxy-2-oxo-[1,3,4]-oxadiazol-3-yl)phenyl]- | 398 | 397.3 |
| | 4-trifluoromethylbenzamide | | |
| 119 | 1-Hexyl-3-[3-(5-methoxy-2-oxo-[1,3,4]-oxadiazol-3-yl)phenyl]urea | 335 | 334.4 |
| 120 | 1-(4-Bromophenyl)-3-[3-(5-methoxy-2-oxo-[1,3,4]-oxadiazol- | 406 | 405.2 |
| | 3-yl)phenyl]urea | | |
| 121 | 1-[3-(5-Methoxy-2-oxo-[1,3,4]-oxadiazol-3-yl)phenyl]-3-(2- | 357 | 356.3 |
| | methoxyphenyl)urea | | |
| 122 | Ethyl 2-[3-[3-(5-methoxy-2-oxo-[1,3,4]-oxadiazol-3-yl)phenyl]- | 427 | 426.4 |
| | ureido]-3-phenylpropionate | | |
| 123 | 1-(2,6-Diisopropylphenyl)-3-[3-(5-methoxy-2-oxo-[1,3,4]-oxadiazol- | 411 | 410.5 |
| | 3-yl)phenyl]urea | | |
| 124 | 1-[3-(5-Methoxy-2-oxo-[1,3,4]-oxadiazol-3-yl)phenyl]-3-octylurea | 363 | 362.4 |
| 125 | 1-(4-Fluorobenzyl)-3-[3-(5-methoxy-2-oxo-[1,3,4]-oxadiazol-3-yl)- | 359 | 358.3 |
| | phenyl]urea | | |
| 126 | 1-(2-Ethylphenyl)-3-[3-(5-methoxy-2-oxo-[1,3,4]-oxadiazol-3-yl)- | 355 | 354.4 |
| | phenyl]urea | | |
| 127 | Ethyl 6-[3-[3-(5-methoxy-2-oxo-[1,3,4]-oxadiazol-3-yl)phenyl]- | 393 | 392.4 |
| | ureido]hexanoate | | |
| 128 | 1-(2,6-Dimethoxyphenyl)-3-[3-(5-methoxy-2-oxo-[1,3,4]-oxadiazol- | 387 | 386.4 |
| | 3-yl)phenyl]urea | | |
| 129 | 5-Methoxy-3-[4-[(thiophen-3-ylmethyl)amino]phenyl]-3H-(1,3,4)- | 304 | 303.3 |
| | oxadiazol-2-one | | |
| 130 | 4-[[4-(5-Methoxy-2-oxo-[1,3,4]-oxadiazol-3- | 437 | 436.3 |
| | yl)phenylamino]methyl]-benzonitrile trifluoroacetate | | |
| 131 | 3-[4-(2-Bromo-4,5-dimethoxybenzylamino)phenyl]-5-methoxy- | 437 | 436.3 |
| | 3H-(1,3,4)-oxadiazol-2-one | | |
| 132 | 3-[4-(3-Ethoxy-4-methoxybenzylamino)phenyl]-5-methoxy- | 486 | 485.4 |
| | 3H-(1,3,4)-oxadiazol-2-one trifluoroacetate | | |
| 133 | Methyl 4-[[4-(5-methoxy-2-oxo-[1,3,4]-oxadiazol-3- | 470 | 469.4 |
| | yl)phenylamino]methyl]benzoate trifluoroacetate | | |
| 134 | 4-[[4-(5-Methoxy-2-oxo-[1,3,4]-oxadiazol-3-yl)phenylamino]- | 356 | 355.3 |
| | methyl]phenyl acetate | | |
| 135 | 5-Methoxy-3-[4-(pentafluorophenylmethylamino)phenyl]- | 388 | 387.3 |
| | 3H-(1,3,4)-oxadiazol-2-one | | |
| 136 | 3-[4-(4-Benzyloxybenzylamino)phenyl]-5-methoxy-3H-(1,3,4)- | 518 | 517.5 |
| | oxadiazol-2-one trifluoroacetate | | |
| 137 | 3-[4-(3,3-Dichlorononylamino)phenyl]-5-methoxy-3H-(1,3,4)- | 517 | 516.3 |
| | oxadiazol-2-one trifluoroacetate | | |
| 138 | 2-[[4-(5-Methoxy-2-oxo-[1,3,4]oxadiazol-3-yl)phenylamino]- | 323 | 322.3 |
| | methyl]benzonitrile | | |
| 139 | 3-[4-(Cyclohexylmethylamino)phenyl]-5-methoxy-3H-(1,3,4)- | 304 | 303.4 |
| | oxadiazol-2-one | | |
| 140 | 5-Methoxy-3-[4-(2,3,5-trichlorobenzylamino)phenyl]-3H-(1,3,4)- | 515 | 514.7 |
| | oxadiazol-2-one trifluoroacetate | | |
| 141 | 3-[4-(5-Bromo-2-fluorobenzylamino)phenyl]-5-methoxy- | 509 | 508.2 |
| | 3H-(1,3,4)oxadiazol-2-one trifluoroacetate | | |
| 142 | 3-[4-(4-Hexyloxybenzylamino)phenyl]-5-methoxy- | 512 | 511.5 |
| | 3H-(1,3,4)oxadiazol-2-one trifluoroacetate | | |
| 143 | 5-Methoxy-3-[4-[3-(3-trifluoromethylphenoxy)benzylamino]phenyl]- | 572 | 571.4 |
| | 3H-(1,3,4)-oxadiazol-2-one trifluoroacetate | | |
| 144 | 3-[4-[(2-Chloroquinolin-3-ylmethyl)amino]phenyl]-5-methoxy- | 497 | 496.8 |
| | 3H-(1,3,4)oxadiazol-2-one trifluoroacetate | | |
| 145 | Methyl 3-methoxy-5-[[4-(5-methoxy-2-oxo-[1,3,4]-oxadiazol- | 501 | 500.4 |
| | 3-yl)phenylamino]methyl]pyridine-2-carboxylate trifluoroacetate | | |
| 146 | 4-[[4-(5-Methoxy-2-oxo-[1,3,4]-oxadiazol-3-yl)phenylamino]- | 454 | 453.5 |
| | methyl]phenyl benzenesulfonate | | |
| 147 | 2-(2,6-Dimethyl-4-methylsulfanylphenoxy)-N-[3-(5-methoxy-2-oxo- | 416 | 415.5 |
| | [1,3,4]-oxadiazol-3-yl)phenyl]acetamide | | |
| 148 | 1-(2,4-Difluorophenyl)-3-[4-(5-methoxy-2-oxo-[1,3,4]-oxadiazol- | 363 | 362.3 |
| | 3-yl)phenyl]urea | | |
| 149 | 1-[4-(5-Methoxy-2-oxo-[1,3,4]-oxadiazol-3-yl)phenyl]- | 419 | 418.4 |
| | 3-(4-phenoxyphenyl)urea | | |
| 150 | 1-(2,6-Difluorophenyl)-3-[4-(5-methoxy-2-oxo-[1,3,4]-oxadiazol- | 363 | 362.3 |
| | 3-yl)phenyl]urea | | |
| 151 | 1-Butyl-3-[4-(5-methoxy-2-oxo-[1,3,4]-oxadiazol-3-yl)phenyl]urea | 307 | 306.3 |
| 152 | 1-(2-Ethoxyphenyl)-3-[4-(5-methoxy-2-oxo-[1,3,4]-oxadiazol- | 371 | 370.4 |
| | 3-yl)phenyl]urea | | |
| 153 | 1-(2,6-Dibromo-4-fluorophenyl)-3-[4-(5-methoxy-2-oxo-[1,3,4]- | 503 | 502.1 |
| | oxadiazol-3-yl)phenyl]urea | | |
| 154 | 1-(4-Butoxyphenyl)-3-[4-(5-methoxy-2-oxo-[1,3,4]-oxadiazol- | 399 | 398.4 |
| | 3-yl)phenyl]urea | | |
| 155 | 1-[4-(5-Methoxy-2-oxo-[1,3,4]-oxadiazol-3-yl)phenyl]- | 411 | 410.3 |
| | 3-(4-trifluoromethoxyphenyl)urea | | |
| 156 | 1-Benzyl-3-[4-(5-methoxy-2-oxo-[1,3,4]-oxadiazol-3-yl)phenyl]urea | 341 | 340.3 |
| 157 | 1-(3-Fluorophenyl)-3-[4-(5-methoxy-2-oxo-[1,3,4]-oxadiazol- | 345 | 344.3 |
| | 3-yl)phenyl]urea | | |
| 158 | Ethyl 6-[3-[4-(5-methoxy-2-oxo-[1,3,4]-oxadiazol-3-yl)phenyl]- | 393 | 392.4 |
| | ureido]hexanoate | | |
| 159 | 1-Biphenyl-4-yl-3-[4-(5-methoxy-2-oxo-[1,3,4]-oxadiazol- | 403 | 402.4 |
| | 3-yl)phenyl]urea | | |
| 160 | Butyl 2-[3-[4-(5-methoxy-2-oxo-[1,3,4]-oxadiazol-3-yl)phenyl]- | 427 | 426.4 |
| | ureido]benzoate | | |
| 161 | 5-Methoxy-3-[3-(7-methoxy-3,7-dimethyloctylamino)phenyl]- | 492 | 491.5 |
| | 3H-[1,3,4]-oxadiazol-2-one trifluoroacetate | | |
| 162 | 5-Methoxy-3-[3-[(thiophen-2-ylmethyl)amino]phenyl]-3H-(1,3,4)- | 418 | 417.4 |
| | oxadiazol-2-one trifluoroacetate | | |
| 163 | 3-(3-Hexylaminophenyl)-5-methoxy-3H-(1,3,4)oxadiazol-2-one | 406 | 405.4 |
| | trifluoroacetate | | |
| 164 | 5-Methoxy-3-[3-(3-phenylpropylamino)phenyl]-3H-(1,3,4)- | 440 | 439.4 |
| | oxadiazol-2-one trifluoroacetate | | |
| 165 | 5-Methoxy-3-(3-undecylaminophenyl)-3H-(1,3,4)oxadiazol-2-one | 476 | 475.5 |
| | trifluoroacetate | | |
| 166 | 5-Methoxy-3-[3-[3-(3-trifluoromethylphenoxy)benzylamino]phenyl]- | 572 | 571.4 |
| | 3H-(1,3,4)-oxadiazol-2-one trifluoroacetate | | |
| 167 | 3-[3-[(2-Chloroquinolin-3-ylmethyl)amino]phenyl]-5-methoxy- | 497 | 496.8 |
| | 3H-(1,3,4)-oxadiazol-2-one trifluoroacetate | | |
| 168 | 4-[[3-(5-Methoxy-2-oxo-[1,3,4]-oxadiazol-3-yl)phenylamino]- | 586 | 585.5 |
| | methyl]phenyl 4-fluorobenzenesulfonate trifluoroacetate | | |
| 169 | 5-Methoxy-3-[3-(3,4,5-trifluorobenzylamino)phenyl]-3H-(1,3,4)- | 466 | 465.3 |
| | oxadiazol-2-one trifluoroacetate | | |
| 170 | 3-[3-(3,5-Bistrifluoromethylbenzylamino)phenyl]-5-methoxy- | 548 | 547.3 |
| | 3H-(1,3,4)-oxadiazol-2-one trifluoroacetate | | |
| 171 | 3-(3-Dec-4-enylaminophenyl)-5-methoxy-3H-(1,3,4)-oxadiazol- | 460 | 459.5 |
| | 2-one trifluoroacetate | | |
| 172 | 3-[3-(3-Cyclopentyl-2-phenethyloxybenzylamino)phenyl]- | 600 | 599.6 |
| | 5-methoxy-3H-(1,3,4)-oxadiazol-2-one trifluoroacetate | | |
| 173 | 4-[[3-(5-Methoxy-2-oxo-(1,3,4)-oxadiazol-3- | 437 | 436.3 |
| | yl)phenylamino]methyl]benzonitrile trifluoroacetate | | |
| 174 | 5-Methoxy-3-[3-[(6-methylpyridin-2-ylmethyl)amino]phenyl]- | 427 | 426.3 |
| | 3H-(1,3,4)oxadiazol-2-one trifluoroacetate | | |
| 175 | 3-[3-(2-Benzyloxyethylamino)phenyl]-5-methoxy-3H-(1,3,4)- | 456 | 455.4 |
| | oxadiazol-2-one trifluoroacetate | | |
| 176 | 3-[3-(2,6-Difluorobenzylamino)phenyl]-5-methoxy-3H-(1,3,4)- | 448 | 447.3 |
| | oxadiazol-2-one trifluoroacetate | | |

| | | m.p. ° C. |
|---|---|---|
| 177 | Dodecanoic acid [4-(5-methoxy-2-oxo-[1,3,4]-oxadiazol-3- | 93 |
| | yl)phenyl]amide | |
| 178 | Octadec-9-enoic acid [4-(5-methoxy-2-oxo-[1,3,4]-oxadiazol-3-yl)phenyl]- | 67 |
| | amide | |
| 179 | 2-Methoxyethyl [4-(5-methoxy-2-oxo-[1,3,4]-oxadiazol-3-yl)-2- | 117 |
| | methylphenyl]carbamate | |
| 180 | 1-(4-Hydroxycyclohexyl)-3-[4-(5-methoxy-2-oxo-[1,3,4]-oxadiazol-3-yl)- | 220 |
| | 2-methylphenyl]urea | |
| 181 | 1,1-Dibutyl-3-[4-(5-methoxy-2-oxo-[1,3,4]-oxadiazol-3-yl)-2-methyl- | Oil |
| | phenyl]urea | |
| 182 | 5-Methoxybenzofuran-2-carboxylic acid [4-(5-methoxy-2-oxo-[1,3,4]- | 199 |
| | oxadiazol-3-yl)-2-methylphenyl]amide | |
| 183 | 4-Methylpiperazine-1-carboxylic acid [4-(5-methoxy-2-oxo-[1,3,4]- | Oil |
| | oxadiazol-3-yl)-2-methylphenyl]amide | |
| 184 | 1-Methylpiperidin-4-yl [4-(5-methoxy-2-oxo-[1,3,4]-oxadiazol-3-yl)- | 235 |
| | 2-methylphenyl]carbamate | |
| 185 | Cyclohexyl [4-(5-methoxy-2-oxo-[1,3,4]-oxadiazol-3-yl)-2-methylphenyl]- | 163 |
| | carbamate | |
| 186 | 4-Benzylpiperidine-1-carboxylic acid [4-(5-methoxy-2-oxo-[1,3,4]- | 146 |
| | oxadiazol-3-yl)-2-methylphenyl]amide | |
| 187 | 1-(2-Diisopropylaminoethyl)-3-[4-(5-methoxy-2-oxo-[1,3,4]-oxadiazol- | 136 |
| | 3-yl)-2-methylphenyl]urea | |
| 188 | 4-(2-{3-[4-(5-Methoxy-2-oxo-[1,3,4]-oxadiazol-3-yl)-2-methylphenyl]- | 200 |
| | ureido}ethyl)benzenesulfonamide | |
| 189 | 1-(1-Benzylpiperidin-4-yl)-3-[4-(5-methoxy-2-oxo-[1,3,4]-oxadiazol-3-yl)- | 198 |
| | 2-methylphenyl]urea | |
| 190 | 1-(4-Isopropylphenyl)-3-[4-(5-methoxy-2-oxo-[1,3,4]-oxadiazol-3-yl)- | 200 |
| | 2-methylphenyl]urea | |
| 191 | 2-{3-[4-(5-Methoxy-2-oxo-[1,3,4]-oxadiazol-3-yl)-2-methylphenyl]ureido}- | 246 |
| | 3-methylbutyric acid | |
| 192 | 1,2,3,4-Tetrahydronaphth-1-yl [4-(5-methoxy-2-oxo-[1,3,4]-oxadiazol- | 159 |
| | 3-yl)-2-methylphenyl]carbamate | |
| 193 | 1-Phenylethyl [4-(5-methoxy-2-oxo-[1,3,4]-oxadiazol-3-yl)-2-methyl- | Oil |
| | phenyl]carbamate | |
| 194 | 4-Isopropylbenzyl [4-(5-methoxy-2-oxo-[1,3,4]-oxadiazol-3-yl)- | 88 |
| | 2-methylphenyl]carbamate | |
| 195 | 4-Trifluoromethoxybenzyl [4-(5-methoxy-2-oxo-[1,3,4]-oxadiazol-3-yl)- | 82 |
| | 2-methylphenyl]carbamate | |
| 196 | 3,5-Dichlorobenzyl [4-(5-methoxy-2-oxo-[1,3,4]-oxadiazol-3-yl)-2- | 169 |
| | methylphenyl]carbamate | |
| 197 | Biphenyl-2-ylmethyl [4-(5-methoxy-2-oxo-[1,3,4]-oxadiazol-3-yl)- | 138 |
| | 2-methylpheny]carbamate | |
| 198 | 5-Chlorobenzofuran-2-carboxylic acid-[4-(5-methoxy-2-oxo-[1,3,4]- | 210 |
| | oxadiazol-3-yl)-2-methylphenyl]amide | |
| 199 | 5-Chlorobenzofuran-2-carboxylic acid [4-(5-methoxy-2-oxo-[1,3,4]- | 209 |
| | oxadiazol-3-yl)phenyl]amide | |

### Example 200

4-Fluorobenzenesulfonic acid morpholide (intermediate)

20 g of morpholine were added dropwise to a solution of 19.5 g 4-fluorobenzenesulfonyl chloride in 100 mL of toluene cooled in ice and the mixture was heated to reflux for 1 hour. After cooling, it was concentrated in vacuo and stirred with water, and the precipitate was filtered off with suction, washed with water and recrystallized from isopropanol.

Yield:16.9 g, melting point: 140° C.

### Example 201

4-Hydrazinobenzenesulfonic acid morpholide (intermediate)

5 g of 4-fluorobenzenesulfonic acid morpholide were dissolved in 15 mL of N-methylpyrrolidone and, after addition of 2.5 g of hydrazine hydrate, heated at 100° C. for 1 hour. After cooling to room temperature, 75 mL of water were added and the mixture was stirred at room temperature. After 2 hours, the solid was filtered off with suction and recrystallized from isopropanol.

Yield: 3.2 g, melting point: 164° C.

The following example was prepared analogously:

### Example 202

4-Hydrazinobenzenesulfonic acid (3,3,5-trimethylcyclohexyl)amide (intermediate)

melting point: 129° C.

### Example 203

4-(3,3,5,5-Tetramethylcyclohexyloxy)nitrobenzene (intermediate)

1.3 g of sodium hydride are added to a solution of 7.8 g of 3,3,5,5-tetramethylcyclohexanol in 50 mL of dimethylformamide, and the mixture is stirred at 40-50° C. for 30 min. Then a total of 7.0 g of 4-fluoronitrobenzene is added in portions, and the mixture is then heated at 100° C. for 3 hours and cooled to room temperature. Addition of 250 mL of ice-water is followed by stirring, and the solid which has formed is filtered off with suction and dried in vacuo.

Yield: 8.6 g, melting point: 70° C.

### Example 204

4-(3,3,5,5-Tetramethylcyclohexyloxy)aniline (intermediate) 8.3 g of 4-(3,3,5,5-tetramethylcyclohexyloxy)nitrobenzene are hydrogenated in 500 mL of methanol in the presence of 400 mg of platinum dioxide under atmospheric pressure until hydrogen uptake ceases. After removal of the catalyst by filtration, the solution is evaporated in a rotary evaporator, and the residue, a gradually solidifying brownish oil, is used without further purification for further reactions.

Yield: 7.3 g

### Example 205

4-(3,3,5,5-Tetramethylcyclohexyloxy)phenylhydrazinehydrochloride (intermediate)

A solution of 1.13 g of sodium nitrite in 7.5 mL of water is added dropwise to a stirred mixture, cooled to −10° C., consisting of 3.7 g of 4-(3,3,5,5-tetramethylcyclohexyloxy)aniline, 7.5 mL of water and 15.5 mL of concentrated HCl, and the mixture is then stirred at −10° C. for 45 min and subsequently added dropwise to a suspension of 9.3 g of tin dichloride dihydrate in 7 mL of concentrated HCl. The precipitate is filtered off with suction, washed with water, suspended in 200 mL of water under nitrogen and decomposed with 100 mL of 30% strength sodium hydroxide solution at 10-15° C. The new precipitate which forms is filtered off with suction, washed with water, taken up in 200 mL of ether and dried with sodium sulfate. The product is then precipitated with ethereal HCl, filtered off with suction and dried in vacuo.

Yield: 2.1 g, melting point: 171° C.

### Example 206

Ethyl N′-(4-morpholinosulfonylphenyl)hydrazinoformate (intermediate)

114 mg of ethyl chloroformate were cautiously added dropwise to a mixture consisting of 0.275 g of 4-hydrazinobenzenesulfonic acid morpholide, 5 mL of methylene chloride and 1 mL of pyridine while cooling in ice, and the mixture was then stirred while slowly warming to RT. After dilution with 10 mL of water, the product was extracted with ethyl acetate, and the ethyl acetate phase was washed several times with water, dried over sodium sulfate and concentrated. The oily crude product obtained in this way was reacted further without further purification.

Yield: 0.25 g

### Example 207

3-(4-Morpholinosulfonylphenyl)-5-ethoxy-3H-(1,3,4)-oxadiazol-2-one

The oil from Example 206 was taken up in 5 mL of methylene chloride and, while stirring and cooling in ice, 1 mL of a 20% strength solution of phosgene in toluene was added. After standing at room temperature overnight, this mixture was diluted with a further 10 mL of methylene chloride and then washed 3 times with water. After drying over sodium sulfate, the mixture was concentrated in vacuo, and the product was purified by column chromatography (silica gel, solvents: methanol:methylene chloride=2:98).

Yield:130 mg, melting point: 195° C.

The following examples were prepared in analogy to Example 207:

### Example 208

3-(4-Morpholinosulfonylphenyl)-5-methoxy-3H-(1,3,4)-oxadiazol-2-one

melting point: 164° C.

### Example 209

3-(4-Trifluoromethoxyphenyl)-5-methoxy-3H-(1,3,4)-oxad iazol-2-one

melting point: 52° C.

### Example 210

3-(4-Trifluoromethoxyphenyl)-5-ethoxy-3H-(1,3,4)-oxad iazol-2-one

melting point: 63° C.

### Example 211

3-(4-Trifluoromethoxyphenyl)-5-isopropoxy-3H-(1,3,4)-oxad iazol-2-one

melting point: oil

### Example 212

3-(4-Trifluoromethoxyphenyl)-5-butoxy-3H-(1,3,4)-oxadiazol-2-one

melting point: oil

### Example 213

3-(4-Trifluoromethoxyphenyl)-5-benzyloxy-3H-(1,3,4)-oxad iazol-2-one

melting point: oil

### Example 214

3-(4-(3,3,5-Trimethylcyclohexylaminosulfonyl)phenyl)-5-methoxy-3H-(1,3,4)-oxadiazol-2-one

melting point: 164° C.

### Example 215

3-(4-(3,3,5,5-Tetramethylcyclohexyloxy)phenyl)-5-ethoxy-3H-(1,3,4)-oxad iazol-2-one

melting point: 111° C.

### Example 216

3-(3-Benzyloxyphenyl)-5-methoxy-3H-(1,3,4)-oxadiazol-2-one

melting point: oil

### Example 217

3-(3-Benzyloxyphenyl)-5-ethoxy-3H-(1,3,4)-oxad iazol-2-one

melting point: 85° C.

### Example 218

3-(3-Trifluoromethoxyphenyl)-5-ethoxy-3H-(1,3,4)-oxad iazol-2-one

melting point: oil

### Example 219

3-(3-Trifluoromethoxyphenyl)-5-methoxy-3H-(1,3,4)-oxad iazol-2-one

melting point: oil

### Example 220

3-(3-Trifluoromethoxyphenyl)-5-isopropoxy-3H-(1,3,4)-oxadiazol-2-one

melting point: oil

### Example 221

3-(4-(2,2,6,6-Tetramethylpiperidin-4-yl-aminosulfonyl)phenyl)-5-methoxy-3H-(1,3,4)-oxadiazol-2-one

melting point: resin

### Example 222

3-(4-(2,2,6,6-Tetramethylpiperidin-4-ylaminosulfonyl)phenyl)-5-isopropoxy-3H-(1,3,4)-oxadiazol-2-one

melting point: resin

### Example 223

3-(4-(2-(Diisopropylaminoethylyaminosulfonyl)phenyl)-5-methoxy-3H-(1,3,4)-oxadiazol-2-one

melting point: oil

### Example 224

3-(4-(2-(Diisopropylaminoethyl)aminosulfonyl)phenyl)-5-isopropoxy-3H-(1,3,4)-oxadiazol-2-one

melting point: oil

### Example 225

3-(4-(4-Methylpiperazin-1-yl-sulfonyl)phenyl)-5-isopropoxy-3H-(1,3,4)-oxadiazol-2-one

melting point: resin

### Example 226

3-(4-(4-Methylpiperazin-1-yl-sulfonyl)phenyl)-5-methoxy-3H-(1,3,4)-oxadiazol-2-one

melting point: resin

### Example 227

3-(3-(4,4,4-Trifluorobutyloxy)phenyl)-5-ethoxy-3H-(1,3,4)-oxadiazol-2-one

melting point: oil

### Example 228

3-(3-(2-Diethylaminoethyloxy)phenyl)-5-ethoxy-3H-(1,3,4)-oxad iazol-2-one

melting point: resin

### Example 229

3-(4-(4-Chlorophenoxy)phenyl)-5-methoxy-3H-(1,3,4)-oxad iazol-2-one

melting point: 68° C.

### Example 230

3-(4-(4-Chlorophenoxy)phenyl)-5-isopropoxy-3H-(1,3,4)-oxadiazol-2-one

melting point: oil

### Example 231

3-(4-(3,3,5-Trimethylcyclohexylaminosulfonyl)phenyl)-5-isopropoxy-1,3,4-oxad iazol-2-one

melting point: oil

### Example 232

3-(3-Phenoxyphenyl)-5-methoxy-3H-(1,3,4)-oxad iazol-2-one

melting point: 89° C.

### Example 233

3-(3-Phenoxyphenyl)-5-ethoxy-3H-(1,3,4)-oxadiazol-2-one

melting point: 50° C.

### Example 234

3-(3-Phenoxyphenyl)-5-isoproxy-3H-(1,3,4)-oxadiazol-2-one

melting point: 58° C.

### Example 235

3-(4-Phenoxyphenyl)-5-methoxy-3H-(1,3,4)-oxadiazol-2-one

melting point: 83° C.

### Example 236

3-(4-Cyclohexylphenyl)-5-methoxy-3H-(1,3,4)-oxad iazol-2-one

melting point: resin

### Example 237

3-(3-(3,3,5,5-Tetramethylcyclohexyloxy)phenyl)-5-methoxy-3H-(1,3,4)-oxadiazol-2-one

melting point: 68° C.

### Example 238

3-(4-Phenylphenyl)-5-methoxy-3H-(1,3,4)-oxadiazol-2-one

melting point: >260° C. (decomp.)

### Example 239

3-(3-(3-Methylphenoxymethyl)phenyl)-5-methoxy-3H-(1,3,4)-oxadiazol-2-one

melting point: 47° C.

### Example 240

3-(3-Phenylphenyl)-5-methoxy-3H-(1,3,4)-oxadiazol-2-one

melting point: 80° C.

### Example 241

3-(4-(3,3-Dimethylpiperidinocarbonyl)phenyl)-5-methoxy-3H-(1,3,4)-oxadiazol-2-one

melting point: resin

### Example 242

3-(4-(3,3,5,5-Tetramethylcyclohexyloxy)phenyl)-5-isopropoxy-3H-(1,3,4)-oxadiazol-2-one

melting point: resin

The compounds of formula 1 show an inhibitory effect on pancreatic lipase (PL). As PL inhibitors, they are able to prevent absorption of fat consumed with the diet and thus lead to a reduction in the fat uptake and the body weight or prevent an increase in body weight. The compounds of formula 1 are particularly suitable for use in the treatment of obesity and of diabetes mellitus of type 1 and 2.

The activity of the compounds was assayed as follows:

1. Preparation of the substrate:

80 μL of tripalmitin (85 mM in chloroform) are mixed with 5 μL of glycerol tri[9,10(n)-³H]oleate (5 mCi/mL in toluene) in a 12 mL polypropylene vessel. Evaporation in a rotary evaporator (50° C.) and addition of 4 mL of 200 mM Tris/HCl (pH 7.6), 0.8% TX-100 are followed by ultrasound treatment of the mixture (Branson B-12 sonifier, output level 4, 3×2 min with 1 min intervals on ice) until a homogeneous milky suspension is produced.

2. Assay:

Lipase buffer: 80 mM Tris/HCl (pH 7.6), 600 mM NaCl, 8 mM CaCl₂, 8 mM benzamidine, 2 mM Pefabloc (Roche Biochemicals) (add the inhibitors only on the day of the assay)

Pancreatic lipase: Enriched preparation from porcine pancreas (Sigma order No. L-0382) dissolved in lipase buffer (100 000 units/500 μL)

Procedure:

5 μL of test substance (in 100% DMSO) or DMSO (control) are mixed with 10 μL of substrate and 5 μL of lipase (in this sequence) and incubated at 30° C. (Eppendorf Thermomixer, 350 min⁻¹) for 30 min. After addition of 325 μL of methanol/chloroform/n-heptane (10/9/7) and 105 μl of 0.1 M K₂CO₃, 0.1 M H₃BO₃ (pH 10.5 adjusted with 1 M KOH) and vigorous mixing, the phases are separated by centrifugation (8000 rpm, Eppendorf centrifuge, 4° C.). 140 μL portions of the aqueous supernatant (contains the liberated radiolabeled oleate; 70% recovery) are transferred into 20 mL scintillation vials and mixed with 6 mL of scintillation cocktail (Beckman Ready Safe). After vigorously mixing and incubating at room temperature for 2 h, the radioactivity is measured in a liquid scintillation counter (Beckman, L8008, tritium channel with quench curve, measurement time 20 min).

Evaluation:

Substances are routinely tested in each concentration in three independent incubation mixtures each with duplicate determination after phase separation (SD<0.02). Background values (reaction under the same conditions but without lipase) are subtracted from all values (corresponds predominantly to the content of glycerol trioleate or free oleate in the substrate preparation in the aqueous phase, <5% of the radioactivity employed). The inhibition of the pancreatic lipase enzymatic activity by a test substance is determined by comparison with an uninhibited control reaction (presence of lipase=0% inhibition; absence of lipase 100% inhibition in each case after background correction). The IC₅₀ is calculated from an inhibition plot with up to 8 concentrations of the test substance. The software package GRAPHIT (Elsevier-BIOSOFT) is used for curve fitting and IC₅₀ determination.

The compounds of formula 1 showed the following effect in this assay system:

| Compound from | IC-50 |
|---|---|
| Example: | μM |
| 86 | 1.5 |
| 210 | 0.7 |
| 212 | 0.5 |
| 213 | 0.5 |
| 216 | 0.8 |
| 218 | 0.7 |
| 220 | 1.8 |
| 229 | 0.6 |

We claim:

1. A method for inhibiting pancreatic Lipase, in a patient in need thereof, comprising administering to the patient a pharmaceutically effective amount of a compound of formula 1: 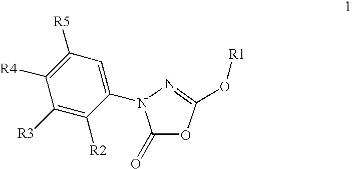
wherein:
R¹ is C₁-C₆-alkyl, or C₃-C₉-cycloalkyl, wherein the alkyl is optionally substituted one or more times by:
hydroxy;
fluorine;
phenyl, optionally substituted one or more times by halogen, C₁-C₉-alkyl, C₁-C₈-alkyloxy, nitro, or CF₃;
C₁-C₄-alkyloxy;
C₁-C₄-alkyl-S—; or
(C₁-C₄-alkyl)₂N—; and
the cycloalkyl is optionally substituted one or more times by:
C₆-C₁₀ aryl, optionally substituted one or more times by halogen, C₁-C₉-alkyl, C₁-C₈-alkyloxy, nitro, or CF₃;
C₁-C₄-alkyl;
C₁-C₄-alkyloxy;
C₁-C₄-alkyl-S—; or
(C₁-C₄-alkyl)₂N—;
R², R³, R⁴ and R⁵ are each, independently,
hydrogen;
halogen;
NO₂;
C₁-C₄-alkyl;
C₁-C₉-alkyloxy, substituted one or more times by fluorine, hydroxy, C₆-C₁₀-aryl, amino, C₁-C₄-alkyl-NH— or (C₁-C₆-alkyl)₂N—;
C₆-C₁₀-aryl-C₁-C₄-alkyloxy, C₆-C₁₀-aryloxy, C₆-C₁₀-aryl, C₆-C₁₀-aryloxy-C₃-C₄-alkyl, C₃-C₈-cycloalkyl or C₃-C₈-cycloalkyloxy, wherein the alkyl is optionally substituted one or more times by halogen, hydroxy, CF₃, (C₁-C₆-alkyl)₂N—, C₁-C₄-alkyloxy or C₁-C₄-alkyl, the aryl is optionally substituted one or more times by halogen, CF₃, C₁-C₈-alkyloxy or C₁-C₉-alkyl, and the cycloalkyl is optionally substituted one or more times by halogen, CF₃, C₁-C₄-alkyloxy, C₆-C₁₀-aryl or C₁-C₄-alkyl;
C₁-C₆-alkyl-NH—SO₂—, wherein the alkyl is optionally substituted by hydroxy, fluorine or (C₁-C₆-alkyl)₂N—;
(2,2,6,6-tetramethylpiperidin-4-yl)-NH—SO₂-;
C₃-C₈-cycloalkyl-NH—SO₂—, wherein the cycloalkyl is optionally substituted one or more times by C₁-C₄-alkyl or C₆-C₁₀-aryl;
(C₁-C₆-alkyl)₂—N—SO₂—;
XCO—;
YSO₂—;
2-oxo-pyrrolidin-1-yl;
2,5-dimethylpyrrol-1-yl; or
R⁷-A-NR⁶,
provided that R², R³, R⁴ and R⁵ are not simultaneously hydrogen;
X is C₁-C₆-alkyloxy;
C₁-C₆-alkyl-NH—;
C₃-C₈-cycloalkyl-NH—;
(C₁-C₆-alkyl)₂N—; or
1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;
Y is 1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;
R⁶ is hydrogen, C₁-C₄-alkyl or C₆-C₁₀-aryl-C₁-C₄-alkyl, wherein the aryl is optionally substituted by halogen, CF₃, C₁-C₈-alkyloxy or C₁-C₉-alkyl;
A is a single bond, —CO—, —O—C(O)—, —SOₙ— or —NR⁸C(O)—;
n is 1 or 2;
R⁷ is hydrogen;
C₁-C₁₈-alkyl or C₂-C₁₈-alkenyl, wherein the alkyl and alkenyl are optionally substituted once to three times by:
C₁-C₄-alkyl;
halogen;
hydroxy;
CF₃;
C₁-C₄-alkyloxy;
(C₁-C₄-alkyl)₂N—;
—COOH;
C₁-C₄-alkyloxycarbonyl;
oxo; or
C₆-C₁₂-aryl, C₆-C₁₂-aryloxy, C₆-C₁₂-arylcarbonyl or C₆-C₁₀-aryl-C₁-C₄-alkyloxy, wherein the aryl is optionally substituted by halogen, C₁-C₉-alkyl, C₁-C₈-alkyloxy, CF₃, aminosulfonyl or methylmercapto;
C₆-C₁₀-aryl-C₁-C₄-alkyl, C₅-C₈-cycloalkyl-C₁-C₄-alkyl, C₅-C₈-cycloalkyl, C₆-C₁₀-aryl-C₂-C₆-alkenyl, C₆-C₁₀-aryl, biphenylyl, biphenylyl-C₁-C₄-alkyl or indanyl, wherein the alkyl, aryl, cycloalkyl, alkenyl, biphenyl and indanyl are each independently optionally substituted one or more times by:
C₁-C₁₈-alkyl, C₁-C₁₈-alkyloxy, C₃-C₈-cycloalkyl, C₁-C₄-alkylcarbonyl, C₆-C₁₀-aryl-C₁-C₄-alkyl, C₆-C₁₀-aryl-C₁-C₄-alkyloxy or C₁-C₆-alkyloxycarbonyl, wherein the alkyl is optionally substituted by fluorine, hydroxy, (C₁-C₄-alkyl)₂N—, C₁-C₄-alkyloxycarbonyl, CF₃ or carboxyl, and the aryl is optionally substituted by halogen, CF₃, C₁-C₉-alkyl or C₁-C₈-alkyloxy;
COOH;
hydroxy;
(C₁-C₄-alkyl)₂N—;
C₆-C₁₀-aryloxy, optionally substituted by C₁-C₉-alkyl, C₁-C₈-alkyloxy, halogen or CF₃;
NO₂;
NC—;
C₆-C₁₀-aryl, optionally substituted by C₁-C₉-alkyl, C₁-C₈-alkyloxy, halogen or CF₃;
fluorosulfonyl;
H₂NSO₂—;
C₁-C₄-alkylcarbonyloxy;
C₆-C₁₀-arylsulfonyloxy;
pyridyl;
C₆-C₁₀-aryl-SO₂NH—;
halogen;
CF₃; or
OCF₃; or
Het-(CH₂)ᵣ—, wherein r is 0, 1, 2 or 3 and Het is saturated or unsaturated 5 to 7-membered heterocycle that is optionally benzo-fused, wherein the heterocycle portion is optionally substituted by:
C₁-C₄-alkyl;
C₆-C₁₀-aryl, optionally substituted by C₁-C₉-alkyl, C₁-C₈-alkyloxy, halogen or CF₃;
halogen;
NO₂;
C₁-C₄-alkyloxy;
C₁-C₄-alkyloxycarbonyl; or
C₆-C₁₀-aryl-C₁-C₄-alkyl or C₆-C₁₀-aryl-C₁-C₄-alkylmercapto, wherein the alkyl is optionally substituted by hydroxy, (C₁-C₄-alkyl)₂N—, fluorine, methoxy or CF₃, and the aryl is optionally substituted by C₁-C₉-alkyl, C₁-C₈-alkyloxy, halogen or CF₃;
and wherein the benzo portion is optionally substituted by halogen, C₁-C₄-alkyloxy or CF₃; and
R⁸ is hydrogen or C₁-C₄-alkyl;
or a prodrug, solvate, pharmacologically acceptable salt, or acid addition salt thereof.

2. The method according to claim 1, wherein: R¹ is C₁-C₆-alkyl, optionally substituted by phenyl.

3. The method according to claim 1, wherein: R⁵ is hydrogen.

4. The method according to claim 1, wherein: R² is hydrogen, halogen, C₁-C₄-alkyl, C₁-C₉-alkyloxy or amino.

5. The method according to claim 1, wherein:
R¹ is C₁-C₆-alkyl, optionally substituted by phenyl;
R⁵ is hydrogen; and
R² is hydrogen, halogen, C₁-C₄-alkyl, C₁-C₉-alkyloxy or amino.

6. The method according to claim 1, wherein:
R³.is hydrogen;
C₁-C₄-alkyl;
C₆-C₁₀-aryl-C₁-C₄-alkyloxy, wherein the aryl is optionally substituted by halogen; or
R⁷-A-N R⁶—;
R⁶ is hydrogen or benzyl;
A is single bond; and
R⁷ is C₆-C₁₀-aryl-C₁-C₄-alkyl, wherein the aryl and alkyl are each independently optionally substituted by halogen, CF₃, cyano, phenyl-C₁-C₄-alkyloxy, CF₃-phenoxy, C₅-C₈-cycloalkyl or fluorosulfonyl;
C₁-C₁₂-alkyl, optionally substituted by C₁-C₄-alkyloxy, phenyl, CF₃ or phenyl-C₁-C₄-alkyloxy;
C₂-C₁₂-alkenyl; or
Het-(CH₂)ᵣ—, wherein r is 0 or 1, and Het is saturated or unsaturated 5 to 7-membered heterocycle that is optionally benzo-fused and wherein the heterocyle portion is optionally substituted by C₁-C₄-alkyl or halogen.

7. The method according to claim 1, wherein:
R² and R³ are each, independently,
hydrogen;
C₆-C₁₀-aryl;
C₃-C₈-cycloalkyl;
optionally C₁-C₄-alkyl-substituted C₆-C₁₀-aryloxymethyl;
optionally mono- or poly-C₁-C₄-alkyl- or halogen-substituted benzyloxy, C₆-C₁₀-aryloxy or C₃-C₈-cycloalkyloxy;
mono- or poly-fluorine-, C₆-C₁₀-aryl- or amino-substituted C₁-C₆-alkyloxy, wherein the amino is optionally substituted once or twice by C₁-C₄-alkyl;
C₁-C₆-alkyl-NH—SO₂—, wherein the alkyl is optionally substituted by (C₁-C₆-alkyl)₂N—;
(2,2,6,6-tetramethylpiperidin-4-yl)-NH—SO₂—;
C₃-C₈-cycloalkyl-NH—SO₂—, wherein the cycloalkyl is optionally substituted by C₁-C₄-alkyl;
(C₁-C₆-alkyl)₂—N—SO₂—;
YSO₂—, wherein Y is 1-piperidinyl, 4-morpholinyl or 1-piperazinyl, wherein the piperidinyl, morpholinyl and piperazinyl are each independently optionally substituted by C₁-C₄-alkyl;
XCO—, wherein X is (C₁-C₆-alkyl)₂N—, 1-piperidinyl, 4-morpholinyl or 1-piperazinyl, wherein the piperidinyl, morpholinyl and piperazinyl are each independently optionally substituted by C₁-C₄-alkyl.

8. The method according to claim 1, wherein:
R⁴ is hydrogen;
2-oxo-pyrrolidin-1-yl;
2,5-dimethylpyrrol-1-yl; or
C₆-C₁₀-aryl-C₁-C₄-alkyloxy, wherein the aryl and alkyl are each independently optionally substituted by halogen.

9. The method according to claim 1, wherein:
R⁴ is R⁷-A-NR⁶;
R⁶ is hydrogen or methyl;
A is single bond; and
R⁷ is hydrogen;
C₁-C₁₂-alkyl, optionally substituted once or twice by halogen;
C₂-C₁₈-alkenyl, optionally substituted once or twice by C₁-C₄-alkyl or C₁-C₄-alkyloxycarbonyl;
C₆-C₁₀-aryl-C₁-C₄-alkyl, wherein the alkyl and aryl are each independently optionally substituted by:
halogen;
C₁-C₆-alkyloxy;
CF₃;
NC—;
C₅-C₆-cycloalkyl;
C₁-C₄-alkyloxycarbonyl;
C₆-C₁₀-aryl-C₁-C₄-alkyl or C₆-C₁₀-aryl-C₁-C₄-alkyloxy, wherein the aryl is optionally substituted by halogen or CF₃;
C₅-C₈-cycloalkyl-C₁-C₄-alkyl; or
Het-(CH₂)ᵣ—, wherein r is 1, 2 or 3 and Het is saturated or unsaturated 5 to 7-membered heterocycle, optionally substituted by halogen, C₁-C₄-alkyloxy or C₁-C₄-alkyloxycarbonyl.

10. The method according to claim 1, wherein:
R⁴ is R⁷-A-NR⁶—;
R⁶ is hydrogen;
A is —CO—; and
R⁷ is C₁-C₁₈-alkyl, optionally substituted by:
halogen;
phenyl;
phenoxy, optionally substituted by methyl, halogen or methylmercapto;
phenylcarbonyl; or
C₁-C₄-alkyloxycarbonyl;
C₂-C₁₈-alkenyl, optionally substituted by C₆-C₁₀-aryl;
C₆-C₁₀-aryl, optionally substituted by:
halogen;
C₁-C₈-alkyl;
phenyl-C₁-C₄-alkyl;
CF₃;
OCF₃;
fluorosulfonyl;
C₁-C₄-alkyloxycarbonyl; or
phenoxy, optionally substituted by C₁-C₄-alkyloxy;
C₆-C₁₀-aryl-C₁-C₄-alkyl, wherein the alkyl is optionally substituted by methoxy or CF₃, and the aryl is optionally substituted by halogen; or
Het-(CH₂)ᵣ—, wherein r is 0 and Het is saturated or unsaturated 5 to 7-membered heterocycle that is optionally benzo-fused, wherein the heterocycle portion is optionally substituted by C₁-C₄-alkyl, halogen, C₁-C₄-alkyloxy, halophenyl or halobenzylmercapto, and wherein the benzo portion is optionally substituted by halogen or methoxy.

11. The method according to claim 1, wherein:
R⁴ is R⁷-A-NR⁶;
R⁶ is hydrogen;
A is —O—C(O)—; and
R⁷ is C₁-C₁₈-alkyl, substituted by CF₃ or phenyl;
C₆-C₁₀-aryl;
C₆-C₁₀-aryl-C₁-C₄-alkyl, wherein the aryl and alkyl are each independently optionally substituted by C₁-C₄-alkyl, halogen, CF₃ or OCF₃, benzyloxy or phenyl; or
Het-(CH₂)ᵣ—, wherein r is 0 or 1 and Het is saturated or unsaturated 5 to 7-membered heterocycle that is optionally benzo-fused, and wherein the heterocycle portion is optionally substituted by C₁-C₄-alkyl or benzyl.

12. The method according to claim 1, wherein:
R⁴ is R⁷-A-NR⁶;
R⁶ is hydrogen;
A is —SO₂—; and
R⁷ is C₁-C₆-alkyl, optionally substituted by CF₃;
C₂-C₄-alkenyl, optionally substituted by phenyl;
C₆-C₁₀-aryl, optionally substituted by C₁-C₆-alkyl, halogen, C₁-C₄-alkyloxy or benzyl;
biphenylyl-C₁-C₄-alkyl, wherein the phenyl and alkyl are optionally substituted by halogen; or
Het-(CH₂)ᵣ—, wherein r is 0 and Het is saturated or unsaturated 5 to 7-membered heterocycle.

13. The method according to claim 1, wherein:
R⁴ is R⁷-A-NR⁶;
R⁶ is hydrogen;
A is —NHCO—; and
R⁷ is C₁-C₁₀-alkyl, optionally substituted by:
C₁-C₄-alkyloxycarbonyl;
(C₁-C₄-alkyl)₂N—; or
phenyl, optionally substituted by halogen or aminosulfonyl;
C₆-C₁₀-aryl, optionally substituted by:
C₁-C₆-alkyl, C₁-C₆-alkyloxy, C₁-C₆-alkyloxycarbonyl, wherein the alkyl is optionally substituted by C₁-C₄-alkyloxycarbonyl or carboxyl;
phenoxy;
OCF₃;
benzyl; or
pyridyl;
C₅-C₈-cycloalkyl, optionally substituted by hydroxy;
indanyl; or
Het-(CH₂)ᵣ-, wherein r is 0 or 1 and Het is saturated or unsaturated 5 to 7-membered heterocycle, optionally substituted by benzyl.

14. The method according to claim 1, wherein:
R² is hydrogen;
R⁵ is hydrogen;
R³ is hydrogen;
C₆-C₁₀-aryl;
C₆-C₁₀-aryloxy;
optionally C₁-C₄-alkyl-substituted C₆-C₁₀-aryloxymethyl;
benzyloxy;
mono- or poly-fluorine- or amino-substituted C₁-C₆-alkyloxy, wherein the amino group is optionally substituted once or twice by times by C₁-C₄-alkyl; or
optionally mono- or poly-C₁-C₄-alkyl-substituted C₃-C₈-cycloalkyloxy; and
R⁴ is hydrogen;
C₆-C₁₀-aryl;
C₃-C₈-cycloalkyl;
optionally mono- or poly-C₁-C₄-alkyl- or halogen-substituted C₆-C₁₀ aryloxy or C₃-C₈-cycloalkyloxy;
mono- or poly-fluorine-substituted C₁-C₆-alkyloxy;
C₁-C₆-alkyl-NH—SO₂—, wherein the alkyl is optionally substituted by (C₁-C₆-alkyl)₂N—;
(2,2,6,6-tetramethylpiperidin-4-yl)-NH—SO₂—;
C₃-C₈-cycloalkyl-NH—SO₂—, wherein the cycloalkyl is optionally substituted one or more times by C₁-C₄-alkyl;
(C₁-C₆-alkyl)₂N—SO₂—;
YSO₂—, wherein Y is 1-piperidinyl, 4-morpholinyl or 1-piperazinyl, wherein the piperidinyl, morpholinyl and piperazinyl are each independently optionally substituted by C₁-C₄-alkyl; or
XCO—, wherein X is (C₁-C₆-alkyl)₂N—, 1-piperidinyl, 4-morpholinyl or 1-piperazinyl, wherein the piperidinyl, morpholinyl and piperazinyl are each independently optionally substituted by C₁-C₄-alkyl.

15. The method according to claim 1, wherein:
R¹ is methyl, ethyl, butyl, isopropyl or benzyl;
R² and R⁵ are hydrogen;
R³ is hydrogen, OCF₃, trifluorobutoxy, 3,3,5,5-tetramethylcyclohexyloxy, benzyloxy, phenoxy, phenyl, 2-diethylamino-ethyloxy or 3-methylphenoxymethyl; and
R⁴ is hydrogen, OCF₃, 3,3,5,5-tetramethylcyclohexyloxy, phenoxy, 4-chlorophenoxy, cyclohexyl, phenyl, morpholinosulfonyl, 3,3,5-trimethylcyclohexylaminosulfonyl, 2,2,6,6-tetramethylpiperidin-4-ylaminosulfonyl, 2-(diisopropylaminoethyl)aminosulfonyl, 4-methylpiperazin-1-ylsulfonyl, 3,3-dimethylpiperidinocarbonyl or 3,5-dichlorophenoxy.

16. The method according to claim 1, wherein:
R¹ is methyl, ethyl, butyl, isopropyl or benzyl;
R² and R⁵ are hydrogen;
R³ is hydrogen, OCF₃, 3,3,5,5-tetramethylcyclohexyloxy, benzyloxy or phenoxy; and
R⁴ is hydrogen, OCF₃, 3,3,5,5-tetramethylcyclohexyloxy, phenoxy, cyclohexyl, phenyl, morpholinosulfonyl or 3,3,5-trimethylcyclohexylaminosulfonyl.

17. The method according to claim 1, wherein:
R¹ is C₁-C₄-alkyl;
R² is hydrogen;
R³ is hydrogen, trifluoromethoxy, benzyloxy;
R⁴ is hydrogen, trifluoromethoxy, 4-chlorophenoxy, 4-trifluoromethylbenzoylamino; and
R⁵ is hydrogen.

18. The method according to claim 1, wherein R¹ is methyl.

19. The method according to claim 1, wherein the compound of formula 1 is:
5-Methoxy-3-(3-benzyloxy-4-(4-trifluoromethylbenzoylamino)phenyl)-3H-(1,3,4)oxadiazol-2-one;
3-(4-Trifluoromethoxyphenyl)-5-ethoxy-3H-(1,3,4)-oxad iazol-2-one;
3-(4-Trifluoromethoxyphenyl)-5-butoxy-3H-(1,3,4)-oxadiazol-2-one;
3-(4-Trifluoromethoxyphenyl)-5-benzyloxy-3H-(1,3,4)-oxad iazol-2-one;
3-(3-Benzyloxyphenyl)-5-methoxy-3H-(1,3,4)-oxad iazol-2-one;
3-(3-Trifluoromethoxyphenyl)-5-ethoxy-3H-(1,3,4)-oxad iazol-2-one;
3-(3-Trifluoromethoxyphenyl)-5-isopropoxy-3H-(1,3,4)-oxadiazol-2-one; or
3-(4-(4-Chlorophenoxy)phenyl)-5-methoxy-3H-(1,3,4)-oxad iazol-2-one.

20. A method for the prophylaxis or treatment of obesity, in a patient in need thereof, comprising administering to the patient a pharmaceutically effective amount of a compound of formula 1: 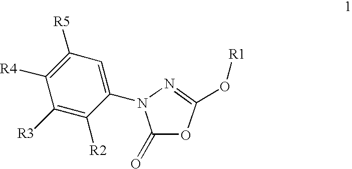
wherein:
R¹ is C₁-C₆-alkyl, or C₃-C₉-cycloalkyl, wherein the alkyl is optionally substituted one or more times by:
hydroxy;
fluorine;
phenyl, optionally substituted one or more times by halogen, C₁-C₉-alkyl, C₁-C₈-alkyloxy, nitro, or CF₃;
C₁-C₄-alkyloxy;
C₁-C₄-alkyl-S—; or
(C₁-C₄-alkyl)₂N—; and
the cycloalkyl is optionally substituted one or more times by:
C₆-C₁₀ aryl, optionally substituted one or more times by halogen, C₁-C₉-alkyl, C₁-C₈-alkyloxy, nitro, or CF₃;
C₁-C₄-alkyl;
C₁-C₄-alkyloxy;
C₁-C₄-alkyl-S—; or
(C₁-C₄-alkyl)₂N—;
R², R³, R⁴ and R⁵ are each, independently,
hydrogen;
halogen;
NO₂;
C₁-C₄-alkyl;
C₁-C₉-alkyloxy, substituted one or more times by fluorine, hydroxy, C₆-C₁₀-aryl, amino, C₁-C₄-alkyl-NH— or (C₁-C₆-alkyl)₂N—;
C₆-C₁₀-aryl-C₁-C₄-alkyloxy, C₆-C₁₀-aryloxy, C₆-C₁₀-aryl, C₆-C₁₀-aryloxy-C₃-C₄-alkyl, C₃-C₈-cycloalkyl or C₃-C₈-cycloalkyloxy, wherein the alkyl is optionally substituted one or more times by halogen, hydroxy, CF₃, (C₁-C₆-alkyl)₂N—, C₁-C₄-alkyloxy or C₁-C₄-alkyl, the aryl is optionally substituted one or more times by halogen, CF₃, C₁-C₈-alkyloxy or C₁-C₉-alkyl, and the cycloalkyl is optionally substituted one or more times by halogen, CF₃ C₁-C₄-alkyloxy, C₆-C₁₀-aryl or C₁-C₄-alkyl;
C₁-C₆-alkyl-NH—SO₂—, wherein the alkyl is optionally substituted by hydroxy, fluorine or (C₁-C₆-alkyl)₂N—;
(2,2,6,6-tetramethylpiperidin-4-yl)-NH—SO₂—;
C₃-C₈-cycloalkyl-NH—SO₂—, wherein the cycloalkyl is optionally substituted one or more times by C₁-C₄-alkyl or C₆-C₁₀-aryl;
(C₁-C₆-alkyl)₂—N—SO₂—;
XCO—;
YSO₂—;
2-oxo-pyrrolidin-1-yl;
2,5-dimethylpyrrol-1-yl; or
R⁷-A-NR⁶—,
provided that R², R³, R⁴ and R⁵ are not simultaneously hydrogen;
X is C₁-C₆-alkyloxy;
C₁-C₆-alkyl-NH—;
C₃-C₈-cycloalkyl-N H—;
(C₁-C₆-alkyl)₂N—; or
1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;
Y is 1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;
R⁶ is hydrogen, C₁-C₄-alkyl or C₆-C₁₀-aryl-C₁-C₄-alkyl, wherein the aryl is optionally substituted by halogen, CF₃, C₁-C₈-alkyloxy or C₁-C₉-alkyl;
A is a single bond, —CO—, —O—C(O)—, —SOₙ— or —NR⁸C(O)—;
n is 1 or 2;
R⁷ is hydrogen;
C₁-C₁₈-alkyl or C₂-C₁₈-alkenyl, wherein the alkyl and alkenyl are optionally substituted once to three times by:
C₁-C₄-alkyl;
halogen;
hydroxy;
CF₃;
C₁-C₄-alkyloxy;
(C₁-C₄-alkyl)₂N—;
—COOH;
C₁-C₄-alkyloxycarbonyl;
oxo; or
C₆-C₁₂-aryl, C₆-C₁₂-aryloxy, C₆-C₁₂-arylcarbonyl or C₆-C₁₀-aryl-C₁-C₄-alkyloxy, wherein the aryl is optionally substituted by halogen, C₁-C₉-alkyl, C₁-C₈-alkyloxy, CF₃, aminosulfonyl or methylmercapto;
C₆-C₁₀-aryl-C₁-C₄-alkyl, C₅-C₈-cycloalkyl-C₁-C₄-alkyl, C₅-C₈-cycloalkyl, C₆-C₁₀-aryl-C₂-C₆-alkenyl, C₆-C₁₀-aryl, biphenylyl, biphenylyl-C₁-C₄-alkyl or indanyl, wherein the alkyl, aryl, cycloalkyl, alkenyl, biphenyl and indanyl are each independently optionally substituted one or more times by:
C₁-C₁₈-alkyl, C₁-C₁₈-alkyloxy, C₃-C₈-cycloalkyl, C₁-C₄-alkylcarbonyl, C₆-C₁₀-aryl-C₁-C₄-alkyl, C₆-C₁₀-aryl-C₁-C₄-alkyloxy or C₁-C₆-alkyloxycarbonyl, wherein the alkyl is optionally substituted by fluorine, hydroxy, (C₁-C₄-alkyl)₂N—, C₁-C₄-alkyloxycarbonyl, CF₃ or carboxyl, and the aryl is optionally substituted by halogen, CF₃, C₁-C₉-alkyl or C₁-C₈-alkyloxy;
COOH;
hydroxy;
(C₁-C₄-alkyl)₂N—;
C₆-C₁₀-aryloxy, optionally substituted by C₁-C₉-alkyl, C₁-C₈-alkyloxy, halogen or CF₃;
NO₂;
NC—;
C₆-C₁₀-aryl, optionally substituted by C₁-C₉-alkyl, C₁-C₈-alkyloxy, halogen or CF₃;
fluorosulfonyl;
H₂NSO₂—;
C₁-C₄-alkylcarbonyloxy;
C₆-C₁₀-arylsulfonyloxy;
pyridyl;
C₆-C₁₀-aryl-SO₂NH—;
halogen;
CF₃; or
OCF₃; or
Het-(CH₂)ᵣ—, wherein r is 0, 1, 2 or 3 and Het is saturated or unsaturated 5 to 7-membered heterocycle that is optionally benzo-fused, wherein the heterocycle portion is optionally substituted by:
C₁-C₄-alkyl;
C₆-C₁₀-aryl, optionally substituted by C₁-C₉-alkyl, C₁-C₈-alkyloxy, halogen or CF₃;
halogen;
NO₂;
C₁-C₄-alkyloxy;
C₁-C₄-alkyloxycarbonyl; or
C₆-C₁₀-aryl-C₁-C₄-alkyl or C₆-C₁₀-aryl-C₁-C₄-alkylmercapto, wherein the alkyl is optionally substituted by hydroxy, (C₁-C₄-alkyl)₂N—, fluorine, methoxy or CF₃, and the aryl is optionally substituted by C₁-C₉-alkyl, C₁-C₈-alkyloxy, halogen or CF₃;
and wherein the benzo portion is optionally substituted by halogen, C₁-C₄-alkyloxy or CF₃; and
R⁸ is hydrogen or C₁-C₄-alkyl;
or a prodrug, solvate, pharmacologically acceptable salt, or acid addition salt thereof.

21. A method for the prophylaxis or treatment of diabetes mellitus of type 1 and 2, in a patient in need thereof, comprising administering to the patient a pharmaceutically effective amount of a compound of formula 1: 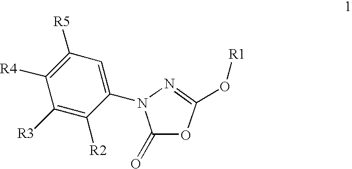
wherein:
R¹ is C₁-C₆-alkyl, or C₃-C₉-cycloalkyl, wherein the alkyl is optionally substituted one or more times by:
hydroxy;
fluorine;
phenyl, optionally substituted one or more times by halogen, C₁-C₉-alkyl, C₁-C₈-alkyloxy, nitro, or CF₃;
C₁-C₄-alkyloxy;
C₁-C₄-alkyl-S—; or
(C₁-C₄-alkyl)₂N—; and
the cycloalkyl is optionally substituted one or more times by:
C₆-C₁₀ aryl, optionally substituted one or more times by halogen, C₁-C₉-alkyl, C₁-C₈-alkyloxy, nitro, or CF₃;
C₁-C₄-alkyl;
C₁-C₄-alkyloxy;
C₁-C₄-alkyl-S—; or
(C₁-C₄-alkyl)₂N—;
R², R³, R⁴ and R⁵ are each, independently,
hydrogen;
halogen;
NO₂;
C₁-C₄-alkyl;
C₁-C₉-alkyloxy, substituted one or more times by fluorine, hydroxy, C₆-C₁₀-aryl, amino, C₁-C₄-alkyl-NH— or (C₁-C₆-alkyl)₂N—;
C₆-C₁₀-aryl-C₁-C₄-alkyloxy, C₆-C₁₀-aryloxy, C₆-C₁₀-aryl, C₆-C₁₀-aryloxy-C₃-C₄-alkyl, C₃-C₈-cycloalkyl or C₃-C₈-cycloalkyloxy, wherein the alkyl is optionally substituted one or more times by halogen, hydroxy, CF₃, (C₁-C₆-alkyl)₂N—, C₁-C₄-alkyloxy or C₁-C₄-alkyl, the aryl is optionally substituted one or more times by halogen, CF₃, C₁-C₈-alkyloxy or C₁-C₉-alkyl, and the cycloalkyl is optionally substituted one or more times by halogen, CF₃ C₁-C₄-alkyloxy, C₆-C₁₀-aryl or C₁-C₄-alkyl;
C₁-C₆-alkyl-NH—SO₂—, wherein the alkyl is optionally substituted by hydroxy, fluorine or (C₁-C₆-alkyl)₂N—;
(2,2,6,6-tetramethylpiperidin-4-yl)-NH—SO₂—;
C₃-C₈-cycloalkyl-NH—SO₂—, wherein the cycloalkyl is optionally substituted one or more times by C₁-C₄-alkyl or C₆-C₁₀-aryl;
(C₁-C₆-alkyl)₂—N—SO₂—;
XCO—;
YSO₂—;
2-oxo-pyrrolidin-1-yl;
2,5-dimethylpyrrol-1-yl; or
R⁷-A-NR⁶
provided that R², R³, R⁴ and R⁵ are not simultaneously hydrogen;
X is C₁-C₆-alkyloxy;
C₁-C₆-alkyl-N H—;
C₃-C₈-cycloalkyl-NH—;
(C₁-C₆-alkyl)₂N—; or
1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;
Y is 1-pyrrolidinyl, 1-piperidinyl, 4-morpholinyl, 4-thiomorpholinyl, or 1-piperazinyl, wherein each is optionally substituted by C₁-C₄-alkyl, benzyl, C₆-C₁₀-aryl, C₁-C₄-alkylcarbonyl, C₆-C₁₀-arylcarbonyl, C₁-C₄-alkyloxycarbonyl, C₁-C₄-alkyl-SO₂— or C₆-C₁₀-aryl-SO₂—;
R⁶ is hydrogen, C₁-C₄-alkyl or C₆-C₁₀-aryl-C₁-C₄-alkyl, wherein the aryl is optionally substituted by halogen, CF₃, C₁-C₈-alkyloxy or C₁-C₉-alkyl;
A is a single bond, —CO—, —O—C(O)—, —SOₙ—or —NR⁸C(O)—;
n is 1 or 2;
R⁷ is hydrogen;
C₁-C₁₈-alkyl or C₂-C₁₈-alkenyl, wherein the alkyl and alkenyl are optionally substituted once to three times by:
C₁-C₄-alkyl;
halogen;
hydroxy;
CF₃;
C₁-C₄-alkyloxy;
(C₁-C₄-alkyl)₂N—;
—COOH;
C₁-C₄-alkyloxycarbonyl;
oxo; or
C₆-C₁₂-aryl, C₆-C₁₂-aryloxy, C₆-C₁₂-arylcarbonyl or C₆-C₁₀-aryl-C₁-C₄-alkyloxy, wherein the aryl is optionally substituted by halogen, C₁-C₉-alkyl, C₁-C₈-alkyloxy, CF₃, aminosulfonyl or methylmercapto;
C₆-C₁₀-aryl-C₁-C₄-alkyl, C₅-C₈-cycloalkyl-C₁-C₄-alkyl, C₅-C₈-cycloalkyl, C₆-C₁₀-aryl-C₂-C₆-alkenyl, C₆-C₁₀-aryl, biphenylyl, biphenylyl-C₁-C₄-alkyl or indanyl, wherein the alkyl, aryl, cycloalkyl, alkenyl, biphenyl and indanyl are each independently optionally substituted one or more times by:
C₁-C₁₈-alkyl, C₁-C₁₈-alkyloxy, C₃-C₈-cycloalkyl, C₁-C₄-alkylcarbonyl, C₆-C₁₀-aryl-C₁-C₄-alkyl, C₆-C₁₀-aryl-C₁-C₄-alkyloxy or C₁-C₆-alkyloxycarbonyl, wherein the alkyl is optionally substituted by fluorine, hydroxy, (C₁-C₄-alkyl)₂N—, C₁-C₄-alkyloxycarbonyl, CF₃ or carboxyl, and the aryl is optionally substituted by halogen, CF₃, C₁-C₉-alkyl or C₁-C₈-alkyloxy;
COOH;
hydroxy;
(C₁-C₄-alkyl)₂N—;
C₆-C₁₀-aryloxy, optionally substituted by C₁-C₉-alkyl, C₁-C₈-alkyloxy, halogen or CF₃;
NO₂;
NC—;
C₆-C₁₀-aryl, optionally substituted by C₁-C₉-alkyl, C₁-C₈-alkyloxy, halogen or CF₃;
fluorosulfonyl;
H₂NSO₂—;
C₁-C₄-alkylcarbonyloxy;
C₆-C₁₀-arylsulfonyloxy;
pyridyl;
C₆-C₁₀-aryl-SO₂NH—;
halogen;
CF₃; or
OCF₃; or
Het-(CH₂)ᵣ—, wherein r is 0, 1, 2 or 3 and Het is saturated or unsaturated 5 to 7-membered heterocycle that is optionally benzo-fused, wherein the heterocycle portion is optionally substituted by:
C₁-C₄-alkyl;
C₆-C₁₀-aryl, optionally substituted by C₁-C₉-alkyl, C₁-C₈-alkyloxy, halogen or CF₃;
halogen;
NO₂;
C₁-C₄-alkyloxy;
C₁-C₄-alkyloxycarbonyl; or
C₆-C₁₀-aryl-C₁-C₄-alkyl or C₆-C₁₀-aryl-C₁-C₄-alkylmercapto, wherein the alkyl is optionally substituted by hydroxy, (C₁-C₄-alkyl)₂N—, fluorine, methoxy or CF₃, and the aryl is optionally substituted by C₁-C₉-alkyl, C₁-C₈-alkyloxy, halogen or CF₃;
and wherein the benzo portion is optionally substituted by halogen, C₁-C₄-alkyloxy or CF₃; and
R⁸ is hydrogen or C₁-C₄-alkyl;
or a prodrug, solvate, pharmacologically acceptable salt, or acid addition salt thereof.
